(19) United States
(12) Patent Application Publication
(10) Pub. No.: US 2021/0280270 A1
(43) Pub. Date: Sep. 9, 2021

(54) METHOD TO DETERMINE IF A CIRCULATING FETAL CELL ISOLATED FROM A PREGNANT MOTHER IS FROM EITHER THE CURRENT OR A HISTORICAL PREGNANCY

(71) Applicants: Illumina, Inc., San Diego, CA (US); Illumina Cambridge Limited, Cambridge (GB)

(72) Inventors: Andrew Craig, Cambridge (GB); Fiona Kaper, Solana Beach, CA (US)

(21) Appl. No.: 17/274,155

(22) PCT Filed: Sep. 6, 2019
(86) PCT No.: PCT/US2019/050078

§ 371 Date: Mar. 5, 2021

Related U.S. Application Data
(60) Provisional application No. 62/728,670, filed on Sep. 7, 2018.

(51) Int. Cl. G16B 20/20; G16B 20/10; G16B 40/20; C12Q 1/6876; G06N 7/00; G06N 20/00; G16H 50/30

(52) U.S. Cl. CPC G16B 20/20; G16B 20/10; G16B 40/20; C12Q 2600/156; G06N 7/005; G06N 20/00; G16H 50/30; C12Q 1/6876

(57) ABSTRACT

Disclosed are methods for determining a genetic origin of fetal cellular DNA obtained from a pregnant female who is carrying a fetus in a current pregnancy. Methods are also disclosed for using the fetal cellular DNA and fetal cell-free DNA (cfDNA) to determine fetal genetic conditions such as copy number variations. The methods disclosed uses a probabilistic model to determine fetal cellular DNA origin based on alleles observed at informative genetic marker of the fetal cellular DNA. Systems and computer program products for performing the methods are also disclosed.

### INCORPORATION BY REFERENCE

A PCT Request Form is filed concurrently with this specification as part of the present application. Each application that the present application claims benefit of or priority to as identified in the concurrently filed PCT Request Form is incorporated by reference herein in its entirety and for all purposes.

### BACKGROUND

The determination of genetic conditions such as copy number variations in a fetus is of important diagnostic value. Previously, most information about copy number, copy number variation (CNV), zygosity, and other genetic conditions of the fetus was provided by cytogenetic resolution that has permitted recognition of structural abnormalities. Conventional procedures for genetic screening and biological dosimetry have utilized invasive procedures, e.g., amniocentesis, cordocentesis, or chorionic villus sampling (CVS), to obtain fetal cells for the analysis of karyotypes. Recognizing the need for more rapid testing methods that do not require cell culture, fluorescence in situ hybridization (FISH), quantitative fluorescence PCR (QF-PCR) and array-Comparative Genomic Hybridization (array-CGH) have been developed as molecular-cytogenetic methods for the analysis of copy number variations. The advent of technologies that allow for sequencing entire genomes in relatively short time, and the discovery of circulating cell-free DNA (cfDNA) including both maternal and fetal DNA in the pregnant mother's blood have provided the opportunity to analyze fetal genetic materials without the risks associated with invasive sampling methods, which provides a tool to diagnose various kinds of copy number variation (CNV) and other properties of genetic sequences of interest.

Diagnosis of fetal genetic conditions using cfDNA in some applications involves heightened technical challenges. In general, fetal cfDNA exists in low fractions relative to maternal cfDNA, typically less than 20%. When the mother is a carrier for a recessive genetic disease, the fetus has a 25% chance of developing the genetic disease if the father is also a carrier. In such case, the mother is heterozygous of the disease related gene, having one disease causing allele and one normal allele; the fetus is homozygous of the disease related gene, having two copies of the disease causing allele. It is desirable to determine if the fetus has inherited genetic disease-causing mutated alleles from both parents in a non-invasive manner using maternal plasma cfDNA. However, it is difficult to differentiate if the fetus is homozygous or heterozygous when the mother is heterozygous using conventional method of non-invasive prenatal diagnosis (NIPD) because the two scenarios have similar sequence tags mapping to the two alleles for a biallelic gene. These challenges underlie the continuing need for noninvasive methods that would reliably diagnose copy number in a variety of clinical settings.

Because of the technical difficulties in using cfDNA for noninvasive prenatal testing (NIPT), various techniques and processes have been developed to increase the sensitivity, selectivity or signal-to-noise ratio of cfDNA-based tests. One way to improve the test is to combine information from fetal cfDNA and fetal cellular DNA to improve the test. In an NIPT, the fetal cellular DNA may be obtained from circulating fetal cells (cFCs), which are fetal cells that originate from a fetus and circulate in a pregnant female carrying the fetus. Typically the cFCs circulate in maternal bodily fluids such as peripheral blood, cervical samples, saliva, sputum, etc. After fetal cellular DNA is obtained, it can be combined with fetal cfDNA to determine genetic conditions of the fetus.

However, fetal cells may persist in maternal blood and other bodily fluids for a long period of time after a pregnancy ends. This means that any fetal cells isolated from a pregnant woman cannot safely be assumed to have originated from the current pregnancy. If the results of prenatal testing are based on a cell originating from a historical pregnancy, this could lead to a serious misdiagnosis.

Embodiments disclosed herein fulfill some of the above needs and in particular offer a means to determine the genetic origin of fetal cellular DNA or cFCs. With the genetic origin known, fetal cellular DNA can then be combined with cfDNA to provide a reliable method that is applicable to the practice of noninvasive prenatal diagnostics.

### SUMMARY

In some embodiments, methods and systems are provided for determining the genetic origin of fetal cellular DNA obtained from a pregnant female who is carrying a fetus in a current pregnancy. The methods are implemented at a computer system that includes one or more processors and system memory.

One aspect of the disclosure relates to a method for determining the genetic origin of fetal cellular DNA obtained from a pregnant female who is carrying a fetus in a current pregnancy. The method includes: (a) receiving a genotype of the fetus in the current pregnancy, wherein the genotype of the fetus in the current pregnancy comprises one or more alleles for each genetic marker of a plurality of genetic markers, where each genetic marker represents a polymorphism at a unique genomic locus (e.g., a unique locus on a reference genome); (b) receiving a genotype of the pregnant female, wherein the genotype of the pregnant female comprises one or more alleles for each genetic marker of the plurality of the genetic markers; (c) identifying, from the genotype of the pregnant female and from the genotype of fetus in the current pregnancy, a set of informative genetic markers, wherein each informative genetic marker of the set of informative genetic markers is homozygous in the pregnant female and is heterozygous in the fetus in the current pregnancy; (d) for the fetal cellular DNA obtained from the pregnant female, determining one or more alleles at each informative genetic marker of the set of informative genetic markers, wherein the fetal cellular DNA originates from the fetus in the current pregnancy or a fetus in a historical pregnancy; (e) providing as input to a probabilistic model the one or more alleles at each informative genetic marker of the fetal cellular DNA obtained from the pregnant female; (f) obtaining as output of the probabilistic model probabilities of three scenarios: the fetal cellular DNA obtained from the pregnant female originates from a fetus in (1) the current pregnancy, (2) the historical pregnancy and having a same father as the fetus in the current pregnancy, and (3) the historical pregnancy and having a different father from the fetus in the current pregnancy; and (g) determining, from the output of the probabilistic model, whether the fetal cellular DNA originates from the fetus in (1) the current pregnancy. At least (e) and (f) are performed by a computer including a processor and memory.

In some implementations, (f) includes: obtaining, as output of the probabilistic model, probabilities of three scenarios: the fetal cellular DNA obtained from the pregnant female originates from a fetus in (1) the current pregnancy, (2) the historical pregnancy and having a same father as the fetus in the current pregnancy, and (3) the historical pregnancy and having a different father from the fetus in the current pregnancy.

In some implementations, (g) includes: determining whether the fetal cellular DNA originates from the fetus in (1) the current pregnancy, (2) the historical pregnancy and having a same father as the fetus in current pregnancy, or (3) the historical pregnancy and having a different father as the fetus in the current pregnancy.

In some implementations, (e) includes providing as input to the probabilistic model a number of shared genetic markers, wherein a shared genetic marker is a genetic marker in the informative genetic markers for which the fetal cellular DNA obtained from the pregnant female and the fetus in the current pregnancy have same alleles.

In some implementations, the probabilistic model calculates the probabilities of the three scenarios given the number of shared genetic markers based on probabilities of the number of shared genetic markers given the three scenarios.

In some implementations, the probabilistic model calculates the probabilities of the three scenarios given the number of shared genetic markers as follows:

$$p ( {s}_{i} \vert k ) = \frac{p ( k \vert {s}_{i} ) p ( {s}_{i} )}{p ( k )}$$

where p(sᵢ|k) is a probability of scenario i, or sᵢ, given the number of shared genetic markers, or k, p(k|sᵢ) is a probability of the number of shared genetic markers given scenario i, p(sᵢ) is an overall probability of scenario i, and p(k) is an overall probability of the number of shared genetic markers.

In some implementations, for each scenario, the probabilistic model simulates the number of shared genetic markers given scenario i, or k|s₁, as a random variable drawn from a beta-binomial distribution.

In some implementations, the probabilistic model simulates the number of shared genetic markers given scenario i, or k|sᵢ as a random variable drawn from a binomial distribution with a success rate and is a random variable drawn from a beta distribution with hyperparameters a₁ and b₁; namely, k|s₁˜BN(n, and μ₁˜Beta(aᵢ,bᵢ), n being the number of informative genetic markers in the set of informative genetic markers.

In some implementations, the probability of the number of shared genetic markers given scenario i is calculated from the following likelihood function:

$$p ( k \vert {s}_{i} ) = ( \begin{matrix}n \\ k\end{matrix} ) \frac{β ( k + {a}_{i} , n - k + {b}_{i} )}{β ( {a}_{i} , {b}_{i} )}$$

Where n is the number of informative genetic markers, k is the number of shared genetic markers, β( ) is a beta function, and aᵢ and bᵢ are the hyperparameters of the beta distribution for scenario i.

In some implementations,

aᵢ=μᵢ*w

bᵢ=(1−μᵢ)*w

wherein w is a parameter representing a number of pseudo counts or observations.

In some implementations, μᵢ is set to correspond to an expected proportion of shared genetic markers among the set of informative genetic markers in scenario i.

In some implementations, the probabilistic model calculates μ₁, the expected proportion of shared genetic markers for scenario (1), as follows:

$${μ}_{1} = 1 - \frac{1}{n + 1}$$

wherein n is the number of informative genetic markers.

In some implementations, the probabilistic model calculates μ₂, the expected proportion of shared genetic markers for scenario (2), as follows,

$${μ}_{2} = \frac{1}{n} {\sum }_{j = 1}^{n} [ {p}_{j} + \frac{1}{2} ( 1 - {p}_{j} ) ]$$

where pⱼ is a population frequency of a hetero-allele at the j^{th} marker, the hetero-allele being an allele at an informative genetic marker found in the fetus in the current pregnancy but not in the pregnant female.

In some implementations, the probabilistic model calculates μ₃, the expected proportion of shared genetic markers for scenario (3), as follows:

$${μ}_{3} = \frac{1}{n} {\sum }_{j = 1}^{n} {p}_{j}$$

where pⱼ is a population frequency of a hetero-allele at the j^{th} marker.

In some implementations, the method further includes providing prior probabilities of the three scenarios to the probabilistic model, wherein the probabilistic model provides posterior probabilities of the three scenarios based on the prior probabilities of the three scenarios, as well as on the alleles at the one or more markers.

In some implementations, the method further includes: obtaining cell free DNA (“cfDNA”) from the pregnant female; and genotyping the cfDNA from the pregnant female to produce (i) the genotype of the fetus in the current pregnancy, and (ii) the genotype of the pregnant female.

In some implementations, the method further includes: obtaining at least one cell of the pregnant female; genotyping cellular DNA obtained from the at least one cell of the pregnant female to produce the genotype of the pregnant female; obtaining cfDNA from the pregnant female; and genotyping the cfDNA from the pregnant female to produce the genotype of the fetus in the current pregnancy.

In some implementations, the fetal cellular DNA is from a circulating fetal cell (“cFC”) circulating in the pregnant female.

In some implementations, the method further includes determining a genetic origin of the cFC.

In some implementations, the fetal cellular DNA is determined to originate from the fetus in the current pregnancy, and the method further includes analyzing the fetal cellular DNA to determine whether the fetus in the current pregnancy has a genetic abnormality.

In some implementations, the genetic abnormality is an aneuploidy.

In some implementations, the analyzing the fetal cellular DNA includes using both information from the fetal cellular DNA and information from fetal cfDNA obtained from the pregnant female during the current pregnancy to determine whether the fetus in the current pregnancy has the genetic abnormality.

In some implementations, each informative genetic marker is biallelic.

Another aspect relates to a computer program product including a non-transitory machine readable medium storing program code that, when executed by one or more processors of a computer system, causes the computer system to implement a method of determining the genetic origin of fetal cellular DNA obtained from a pregnant female who is carrying a fetus in a current pregnancy. The program code includes: (a) code for determining, for the fetal cellular DNA obtained from the pregnant female, one or more alleles at each informative genetic marker of a set of informative genetic markers, wherein each informative genetic marker represents a polymorphism at a unique genomic locus, each informative genetic marker is homozygous in the pregnant female and is heterozygous in the fetus in the current pregnancy, and the fetal cellular DNA originates from the fetus in the current pregnancy or a fetus in a historical pregnancy. The program code also includes (b) code for providing as input to a probabilistic model the one or more alleles at each informative genetic marker of the fetal cellular DNA obtained from the pregnant female; (c) code for obtaining as output of the probabilistic model probabilities of three scenarios: the fetal cellular DNA obtained from the pregnant female originating from a fetus in (1) the current pregnancy, (2) the historical pregnancy and having a same father as the fetus in the current pregnancy, and (3) the historical pregnancy and having a different father from the fetus in the current pregnancy; and (d) code for determining, from the output of the probabilistic model, whether the fetal cellular DNA originates from the fetus in (1) the current pregnancy.

An additional aspect relates to a computer system, including: one or more processors; system memory; and one or more computer-readable storage media having stored thereon computer-executable instructions that, when executed by the one or more processors, cause the computer system to implement a method of determining the genetic origin of fetal cellular DNA obtained from a pregnant female who is carrying a fetus in a current pregnancy. The method includes: (a) determining, for the fetal cellular DNA obtained from the pregnant female, one or more alleles at each informative genetic marker of to set of informative genetic markers, wherein each informative genetic marker represents a polymorphism at a unique genomic locus, each informative genetic marker is homozygous in the pregnant female and is heterozygous in the fetus in the current pregnancy, and the fetal cellular DNA originates from the fetus in the current pregnancy or a fetus in a historical pregnancy; (b) providing as input to a probabilistic model the one or more alleles at each informative genetic marker of the fetal cellular DNA obtained from the pregnant female; (c) obtaining as output of the probabilistic model probabilities of three scenarios: the fetal cellular DNA obtained from the pregnant female originating from a fetus in (1) the current pregnancy, (2) the historical pregnancy and having a same father as the fetus in the current pregnancy, and (3) the historical pregnancy and having a different father from the fetus in the current pregnancy; and (d) determining, from the output of the probabilistic model, whether the fetal cellular DNA originates from the fetus in (1) the current pregnancy.

Another aspect of the disclosure relates to a method for matching pairs of character strings using probabilistic modeling and computer simulation, wherein two character strings in any pair have a same number of characters, the method comprising: (a) receiving a first pair of character strings; (b) receiving a fifth pair of character strings; (c) identifying a set of informative character positions in both the first pair of character strings and the fifth pair of character strings, wherein each informative character position of the set of informative character positions (i) represents a unique position in each character string, (ii) has one or both of two different characters in any pair of character strings, (iii) has only one character of said two different characters in the fifth pair of character strings, and (iv) has both characters of said two different characters in the first pair of character strings; (d) determining, for a fourth pair of character strings, characters at the set of informative character positions; (e) receiving a training dataset comprising pairs of character strings and training a probabilistic model using the training dataset; (f) providing, as input to the probabilistic model, characters at the set of informative character positions of the fourth pair of character strings; (g) obtaining, as output of the probabilistic model, probabilities of three scenarios: the fourth pair of character strings matches the first, a second, and a third pair of character strings, wherein two different character strings of each pair of character strings have a same length, each informative character position has a corresponding position on each character strings, the first pair of character strings is obtainable by recombining the fifth pair of character strings with a sixth pair of pair of character strings, the second pair of character strings is also obtainable by recombining the fifth pair of character strings with the sixth pair of character strings, and the third pair of character strings is obtainable by recombining the fifth pair of character strings with a seventh pair of character strings; and (h) determining, from the output of the probabilistic model, whether the fourth pair of character strings matches the first, second, or third pair of character strings. At least (e), (f), and (g) are performed by a computer system comprising a processor and memory.

In some implementations, wherein (f) includes: obtaining probabilities of three scenarios: the fourth pair of character strings matches the first, a second, and a third pair of character strings, wherein the second pair of character strings is obtainable by recombining the fifth pair of character strings with the sixth pair of character strings, and the third pair of character strings is obtainable by recombining the fifth pair of character strings with a seventh pair of character strings.

In some implementations, wherein (g) includes determining, from the output of the probabilistic model, whether the fourth pair of character strings matches the first, second, or third pair of character strings.

In some implementations, a computer system including one or more processors and system memory is configured to perform any of the methods described above.

An additional aspect of the disclosure relates a computer program product including one or more computer-readable non-transitory storage media having stored thereon computer-executable instructions that, when executed by one or more processors of a computer system, cause the computer system to implement any of the methods above.

Although the examples herein concern humans and the language is primarily directed to human concerns, the concepts described herein are applicable to genomes from any plant or animal. These and other objects and features of the present disclosure will become more fully apparent from the following description and appended claims, or may be learned by the practice of the disclosure as set forth hereinafter.

### INCORPORATION BY REFERENCE

All patents, patent applications, and other publications, including all sequences disclosed within these references, referred to herein are expressly incorporated herein by reference, to the same extent as if each individual publication, patent or patent application was specifically and individually indicated to be incorporated by reference. All documents cited are, in relevant part, incorporated herein by reference in their entireties for the purposes indicated by the context of their citation herein. However, the citation of any document is not to be construed as an admission that it is prior art with respect to the present disclosure.

### BRIEF DESCRIPTION OF THE DRAWINGS

FIG. 1 shows a process for determining a source of circling fetal cells.

FIG. 2 shows a process for determining a source of fetal cellular DNA.

FIG. 3 illustrates a process for determining copy number variation using fetal cellular DNA originating from a fetus of a current pregnancy and fetal cfDNA from said fetus.

FIG. 4 illustrates components of a probabilistic model.

FIG. 5 illustrates a process for matching pairs of character strings using probabilistic modeling and computer simulation.

FIG. 6 shows a process flow of a method for determining a sequence of interest of a fetus.

FIG. 7 depicts a flowchart of a process to obtain mother-and-fetus cfDNA and fetal cellular DNA using a fixed whole blood sample obtained from a pregnant mother.

FIG. 8 illustrates an example process to obtain fetal cellular DNA from fetal NRBCs that have been isolated from maternal cells.

FIG. 9 shows a flowchart of a process for isolating fetal NRBCs from a maternal blood sample.

FIG. 10 illustrates a typical computer system that can serve as a computational apparatus according to certain embodiments.

FIG. 11 shows one implementation of a dispersed system for producing a call or diagnosis from a test sample.

FIG. 12 shows the options for performing various operations at distinct locations according to some implementations of the disclosure.

FIG. 13 illustrates beta distributions of the expected portion of shared genetic markers (p) for three different scenarios.

FIG. 14 illustrates log probability as a function of number of shared/matched genetic markers.

### DETAILED DESCRIPTION

### Definitions

Unless otherwise indicated, the practice of the method and system disclosed herein involves conventional techniques and apparatus commonly used in molecular biology, microbiology, protein purification, protein engineering, protein and DNA sequencing, and recombinant DNA fields, which are within the skill of the art. Such techniques and apparatus are known to those of skill in the art and are described in numerous texts and reference works (See e.g., Sambrook et al., “Molecular Cloning: A Laboratory Manual,” Third Edition (Cold Spring Harbor), [2001]); and Ausubel et al., “Current Protocols in Molecular Biology” [1987]).

Numeric ranges are inclusive of the numbers defining the range. It is intended that every maximum numerical limitation given throughout this specification includes every lower numerical limitation, as if such lower numerical limitations were expressly written herein. Every minimum numerical limitation given throughout this specification will include every higher numerical limitation, as if such higher numerical limitations were expressly written herein. Every numerical range given throughout this specification will include every narrower numerical range that falls within such broader numerical range, as if such narrower numerical ranges were all expressly written herein.

When the term “about” is used to modify a quantity, it refers to a range from the quantity minus 10% to the quantity plus 10%.

The headings provided herein are not intended to limit the disclosure.

Unless defined otherwise herein, all technical and scientific terms used herein have the same meaning as commonly understood by one of ordinary skill in the art. Various scientific dictionaries that include the terms included herein are well known and available to those in the art. Although any methods and materials similar or equivalent to those described herein find use in the practice or testing of the embodiments disclosed herein, some methods and materials are described.

The terms defined immediately below are more fully described by reference to the Specification as a whole. It is to be understood that this disclosure is not limited to the particular methodology, protocols, and reagents described, as these may vary, depending upon the context they are used by those of skill in the art. As used herein, the singular terms “a,” “an,” and “the” include the plural reference unless the context clearly indicates otherwise.

Unless otherwise indicated, nucleic acids are written left to right in 5′ to 3′ orientation and amino acid sequences are written left to right in amino to carboxy orientation, respectively.

Circulating cell-free DNA or simply cell-free DNA (cfDNA) are DNA fragments that are not confined within cells and are freely circulating in the bloodstream or other bodily fluids. It is known that cfDNA have different origins, in some cases from donor tissue DNA circulating in a donee's blood, in some cases from tumor cells or tumor affected cells, in other cases from fetal DNA circulating in maternal blood. In general, cfDNA are fragmented and include only a small portion of a genome, which may be different from the genome of the individual from which the cfDNA is obtained.

The term non-circulating genomic DNA (gDNA) or cellular DNA are used to refer to DNA molecules that are confined in cells and often include a complete genome.

On a general level, the noun “genotype” refers to the genetic constitution of an organism or a cell. More specifically, a genotype may refer to alleles for one or more genetic markers of interest. For example, a genotype for a phenotype of interest may include alleles of multiple genes or genetic markers. A genotype may also refer to alleles of a single gene or a single genetic marker. For instance, a gene may have three different genotypes—AA, aa, and aA. As a verb, “genotyping” refers to an act or a process of determining the genetic constitution of an organism, a cell, or one or more genetic markers.

A beta distribution is a family of continuous probability distributions defined on the interval [0, 1] parameterized by two positive shape parameters, denoted by, e.g., α and β (or a and b), that appear as exponents of the random variable and control the shape of the distribution. The beta distribution has been applied to model the behavior of random variables limited to intervals of finite length in a wide variety of disciplines. In Bayesian inference, the beta distribution is the conjugate prior probability distribution for the Bernoulli, binomial, negative binomial and geometric distributions. For example, the beta distribution can be used in Bayesian analysis to describe initial knowledge concerning probability of success. If a random variable X follows the beta distribution, the random variable X can be denoted as X˜Beta(α, β) or X˜β (a, b).

A binomial distribution is a discrete probability distribution of the number of successes in a sequence of n independent experiments, each asking a yes-no question, and each with its own Boolean-valued outcome: a random variable containing single bit of information: positive (with probability p) or negative (with probability q=1−p). For a single trial, i.e., n=1, the binomial distribution is a Bernoulli distribution. The binomial distribution is frequently used to model the number of successes in a sample of size n drawn with replacement from a population of size N. If a random variable X follows the binomial distribution with parameters n∈ [symbol] and p∈[0,1], the random variable X can be denoted as as X˜B(n,p) or X˜BN(n, p). Put another way, X represents the number of successful trials out of a total of n trials, and p is the probability of each trial yielding a successful result.

A beta-binomial distribution is a binomial distribution BN(n,p) in which the success rate p is a random variable from a beta distribution Beta(a, b). The random variable X can be denoted as X˜BB (n, a, b).

Polymorphism and genetic polymorphism are used interchangeably herein to refer to the occurrence in the same population of two or more alleles at one genomic locus, each with appreciable frequency.

Polymorphism site and polymorphic site are used interchangeably herein to refer to a locus on a genome at which two or more alleles reside. In some implementations, it is used to refer to a single nucleotide variation with two alleles of different bases.

The term “allele count” refers to the count or number of sequence reads of a particular allele. In some implementations, it can be determined by mapping reads to a location in a reference genome, and counting the reads that include an allele sequence and are mapped to the reference genome.

Allele frequency or gene frequency is the frequency of an allele of a gene (or a variant of the gene) relative to other alleles of the gene, which can be expressed as a fraction or percentage. An allele frequency is often associated with a particular genomic locus, because a gene is often located at with one or more locus. However, an allele frequency as used herein can also be associated with a size-based bin of DNA fragments. In this sense, DNA fragments such as cfDNA containing an allele are assigned to different size-based bins. The frequency of the allele in a size-based bin relative to the frequency of other alleles is an allele frequency.

The term “read” refers to a sequence obtained from a portion of a nucleic acid sample. Typically, though not necessarily, a read represents a short sequence of contiguous base pairs in the sample. The read may be represented symbolically by the base pair sequence (in A, T, C, or G) of the sample portion. It may be stored in a memory device and processed as appropriate to determine whether it matches a reference sequence or meets other criteria. A read may be obtained directly from a sequencing apparatus or indirectly from stored sequence information concerning the sample. In some cases, a read is a DNA sequence of sufficient length (e.g., at least about 25 bp) that can be used to identify a larger sequence or region, e.g., that can be aligned and specifically assigned to a chromosome or genomic region or gene.

The term “genomic read” is used in reference to a read of any segments in the entire genome of an individual.

The term “parameter” is used herein represents a physical feature whose value or other characteristic has an impact a relevant condition such as copy number variation. In some cases, the term parameter is used with reference to a variable that affects the output of a mathematical relation or model, which variable may be an independent variable (i.e., an input to the model) or an intermediate variable based on one or more independent variables. Depending on the scope of a model, an output of one model may become an input of another model, thereby becoming a parameter to the other model.

The term “copy number variation” herein refers to variation in the number of copies of a nucleic acid sequence present in a test sample in comparison with the copy number of the nucleic acid sequence present in a reference sample. In certain embodiments, the nucleic acid sequence is 1 kb or larger. In some cases, the nucleic acid sequence is a whole chromosome or significant portion thereof. A “copy number variant” refers to the sequence of nucleic acid in which copy-number differences are found by comparison of a nucleic acid sequence of interest in test sample with an expected level of the nucleic acid sequence of interest. For example, the level of the nucleic acid sequence of interest in the test sample is compared to that present in a qualified sample. Copy number variants/variations include deletions, including microdeletions, insertions, including microinsertions, duplications, multiplications, and translocations. CNVs encompass chromosomal aneuploidies and partial aneuploidies.

The term “aneuploidy” herein refers to an imbalance of genetic material caused by a loss or gain of a whole chromosome, or part of a chromosome.

The terms “chromosomal aneuploidy” and “complete chromosomal aneuploidy” herein refer to an imbalance of genetic material caused by a loss or gain of a whole chromosome, and includes germline aneuploidy and mosaic aneuploidy.

The term “plurality” refers to more than one element. For example, the term is used herein in reference to a number of nucleic acid molecules or sequence tags that are sufficient to identify significant differences in copy number variations in test samples and qualified samples using the methods disclosed herein. In some embodiments, at least about 3×10⁶ sequence tags of between about 20 and 40 bp are obtained for each test sample. In some embodiments, each test sample provides data for at least about 5×10⁶, 8×10⁶, 10×10⁶, 15×10⁶, 20×10⁶, 30×10⁶, 40×10⁶, or 50×10⁶ sequence tags, each sequence tag comprising between about 20 and 40 bp.

The term “paired end reads” refers to reads from paired end sequencing that obtains one read from each end of a nucleic acid fragment. Paired end sequencing may involve fragmenting strands of polynucleotides into short sequences called inserts. Fragmentation is optional or unnecessary for relatively short polynucleotides such as cell free DNA molecules.

The terms “polynucleotide,” “nucleic acid” and “nucleic acid molecules” are used interchangeably and refer to a covalently linked sequence of nucleotides (i.e., ribonucleotides for RNA and deoxyribonucleotides for DNA) in which the 3′ position of the pentose of one nucleotide is joined by a phosphodiester group to the 5′ position of the pentose of the next. The nucleotides include sequences of any form of nucleic acid, including, but not limited to RNA and DNA molecules such as cfDNA molecules. The term “polynucleotide” includes, without limitation, single- and double-stranded polynucleotide.

The term “test sample” herein refers to a sample, typically derived from a biological fluid, cell, tissue, organ, or organism, comprising a nucleic acid or a mixture of nucleic acids comprising at least one nucleic acid sequence that is to be screened for copy number variation. In certain embodiments the sample comprises at least one nucleic acid sequence whose copy number is suspected of having undergone variation. Such samples include, but are not limited to sputum/oral fluid, amniotic fluid, blood, a blood fraction, or fine needle biopsy samples (e.g., surgical biopsy, fine needle biopsy, etc.), urine, peritoneal fluid, pleural fluid, and the like. Although the sample is often taken from a human subject (e.g., patient), the assays can be used to copy number variations (CNVs) in samples from any mammal, including, but not limited to dogs, cats, horses, goats, sheep, cattle, pigs, etc. The sample may be used directly as obtained from the biological source or following a pretreatment to modify the character of the sample. For example, such pretreatment may include preparing plasma from blood, diluting viscous fluids and so forth. Methods of pretreatment may also involve, but are not limited to, filtration, precipitation, dilution, distillation, mixing, centrifugation, freezing, lyophilization, concentration, amplification, nucleic acid fragmentation, inactivation of interfering components, the addition of reagents, lysing, etc. If such methods of pretreatment are employed with respect to the sample, such pretreatment methods are typically such that the nucleic acid(s) of interest remain in the test sample, sometimes at a concentration proportional to that in an untreated test sample (e.g., namely, a sample that is not subjected to any such pretreatment method(s)). Such “treated” or “processed” samples are still considered to be biological “test” samples with respect to the methods described herein.

The term “training set” herein refers to a set of training samples that can comprise affected and/or unaffected samples and are used to develop a model for analyzing test samples. In some embodiments, the training set includes unaffected samples. In these embodiments, thresholds for determining CNV are established using training sets of samples that are unaffected for the copy number variation of interest. The unaffected samples in a training set may be used as the qualified samples to identify normalizing sequences, e.g., normalizing chromosomes, and the chromosome doses of unaffected samples are used to set the thresholds for each of the sequences, e.g., chromosomes, of interest. In some embodiments, the training set includes affected samples. The affected samples in a training set can be used to verify that affected test samples can be easily differentiated from unaffected samples.

A training set is also a statistical sample in a population of interest, which statistical sample is not to be confused with a biological sample. A statistical sample often comprises multiple individuals, data of which individuals are used to determine one or more quantitative values of interest generalizable to the population. The statistical sample is a subset of individuals in the population of interest. The individuals may be persons, animals, tissues, cells, other biological samples (i.e., a statistical sample may include multiple biological samples), and other individual entities providing data points for statistical analysis.

Usually, a training set is used in conjunction with a validation set. The term “validation set” is used to refer to a set of individuals in a statistical sample, data of which individuals are used to validate or evaluate the quantitative values of interest determined using a training set. In some embodiments, for instance, a training set provides data for calculating a mask for a reference sequence, while a validation set provides data to evaluate the validity or effectiveness of the mask.

“Evaluation of copy number” is used herein in reference to the statistical evaluation of the status of a genetic sequence related to the copy number of the sequence. For example, in some embodiments, the evaluation comprises the determination of the presence or absence of a genetic sequence. In some embodiments the evaluation comprises the determination of the partial or complete aneuploidy of a genetic sequence. In other embodiments the evaluation comprises discrimination between two or more samples based on the copy number of a genetic sequence. In some embodiments, the evaluation comprises statistical analyses, e.g., normalization and comparison, based on the copy number of the genetic sequence.

The term “sequence of interest” or “nucleic acid sequence of interest” herein refers to a nucleic acid sequence that is associated with a difference in sequence representation between healthy and diseased individuals. A sequence of interest can be a sequence on a chromosome that is misrepresented, i.e., over- or under-represented, in a disease or genetic condition. A sequence of interest may be a portion of a chromosome, i.e., chromosome segment, or a whole chromosome. For example, a sequence of interest can be a chromosome that is over-represented in an aneuploidy condition, or a gene encoding a tumor-suppressor that is under-represented in a cancer. Sequences of interest include sequences that are over- or under-represented in the total population, or a subpopulation of cells of a subject. A “qualified sequence of interest” is a sequence of interest in a qualified sample. A “test sequence of interest” is a sequence of interest in a test sample.

The term “normalizing sequence” herein refers to a sequence that is used to normalize the number of sequence tags mapped to a sequence of interest associated with the normalizing sequence. In some embodiments, a normalizing sequence comprises a robust chromosome. A “robust chromosome” is one that is unlikely to be aneuploid. In some cases involving the human chromosome, a robust chromosome is any chromosome other than the X chromosome, Y chromosome, chromosome 13, chromosome 18, and chromosome 21. In some embodiments, the normalizing sequence displays a variability in the number of sequence tags that are mapped to it among samples and sequencing runs that approximates the variability of the sequence of interest for which it is used as a normalizing parameter. The normalizing sequence can differentiate an affected sample from one or more unaffected samples. In some implementations, the normalizing sequence best or effectively differentiates, when compared to other potential normalizing sequences such as other chromosomes, an affected sample from one or more unaffected samples. In some embodiments, the variability of the normalizing sequence is calculated as the variability in the chromosome dose for the sequence of interest across samples and sequencing runs. In some embodiments, normalizing sequences are identified in a set of unaffected samples.

A “normalizing chromosome,” “normalizing denominator chromosome,” or “normalizing chromosome sequence” is an example of a “normalizing sequence.” A “normalizing chromosome sequence” can be composed of a single chromosome or of a group of chromosomes. In some embodiments, a normalizing sequence comprises two or more robust chromosomes. In certain embodiments, the robust chromosomes are all autosomal chromosomes other than chromosomes, X, Y, 13, 18, and 21. A “normalizing segment” is another example of a “normalizing sequence.” A “normalizing segment sequence” can be composed of a single segment of a chromosome or it can be composed of two or more segments of the same or of different chromosomes. In certain embodiments, a normalizing sequence is intended to normalize for variability such as process-related, interchromosomal (intra-run), and inter-sequencing (inter-run) variability.

The term “coverage” refers to the abundance of sequence tags mapped to a defined sequence. Coverage can be quantitatively indicated by sequence tag density (or count of sequence tags), sequence tag density ratio, normalized coverage amount, adjusted coverage values, etc.

The term “Next Generation Sequencing (NGS)” herein refers to sequencing methods that allow for massively parallel sequencing of clonally amplified molecules and of single nucleic acid molecules. Non-limiting examples of NGS include sequencing-by-synthesis using reversible dye terminators, and sequencing-by-ligation.

The term “parameter” herein refers to a numerical value that characterizes a property of a system. Frequently, a parameter numerically characterizes a quantitative data set and/or a numerical relationship between quantitative data sets. For example, a ratio (or function of a ratio) between the number of sequence tags mapped to a chromosome and the length of the chromosome to which the tags are mapped, is a parameter.

The terms “threshold value” and “qualified threshold value” herein refer to any number that is used as a cutoff to characterize a sample such as a test sample containing a nucleic acid from an organism suspected of having a medical condition. The threshold may be compared to a parameter value to determine whether a sample giving rise to such parameter value suggests that the organism has the medical condition. In certain embodiments, a qualified threshold value is calculated using a qualifying data set and serves as a limit of diagnosis of a copy number variation, e.g., an aneuploidy, in an organism. If a threshold is exceeded by results obtained from methods disclosed herein, a subject can be diagnosed with a copy number variation, e.g., trisomy 21. Appropriate threshold values for the methods described herein can be identified by analyzing normalized values (e.g. chromosome doses, NCVs or NSVs) calculated for a training set of samples. Threshold values can be identified using qualified (i.e., unaffected) samples in a training set which comprises both qualified (i.e., unaffected) samples and affected samples. The samples in the training set known to have chromosomal aneuploidies (i.e., the affected samples) can be used to confirm that the chosen thresholds are useful in differentiating affected from unaffected samples in a test set (see the Examples herein). The choice of a threshold is dependent on the level of confidence that the user wishes to have to make the classification. In some embodiments, the training set used to identify appropriate threshold values comprises at least 10, at least 20, at least 30, at least 40, at least 50, at least 60, at least 70, at least 80, at least 90, at least 100, at least 200, at least 300, at least 400, at least 500, at least 600, at least 700, at least 800, at least 900, at least 1000, at least 2000, at least 3000, at least 4000, or more qualified samples. It may be advantageous to use larger sets of qualified samples to improve the diagnostic utility of the threshold values.

The term “bin” refers to a segment of a sequence or a segment of a genome. In some embodiments, bins are contiguous with one another within the genome or chromosome. Each bin may define a sequence of nucleotides in a reference sequence such as a reference genome. Sizes of the bin may be 1 kb, 100 kb, 1 Mb, etc., depending on the analysis required by particular applications and sequence tag density. In addition to their positions within a reference sequence, bins may have other characteristics such as sample coverage and sequence structure characteristics such as G-C fraction.

The term “read” refers to a sequence obtained from a portion of a nucleic acid sample. Typically, though not necessarily, a read represents a short sequence of contiguous base pairs in the sample. The read may be represented symbolically by the base pair sequence (in A, T, C, or G) of the sample portion. It may be stored in a memory device and processed as appropriate to determine whether it matches a reference sequence or meets other criteria. A read may be obtained directly from a sequencing apparatus or indirectly from stored sequence information concerning the sample. In some cases, a read is a DNA sequence of sufficient length (e.g., at least about 25 bp) that can be used to identify a larger sequence or region, e.g., that can be aligned and specifically assigned to a chromosome or genomic region or gene.

The term “genomic read” is used in reference to a read of any segments in the entire genome of an individual.

The term “sequence tag” is herein used interchangeably with the term “mapped sequence tag” to refer to a sequence read that has been specifically assigned, i.e., mapped, to a larger sequence, e.g., a reference genome, by alignment. Mapped sequence tags are uniquely mapped to a reference genome, i.e., they are assigned to a single location to the reference genome. Unless otherwise specified, tags that map to the same sequence on a reference sequence are counted once. Tags may be provided as data structures or other assemblages of data. In certain embodiments, a tag contains a read sequence and associated information for that read such as the location of the sequence in the genome, e.g., the position on a chromosome. In certain embodiments, the location is specified for a positive strand orientation. A tag may be defined to allow a limited amount of mismatch in aligning to a reference genome. In some embodiments, tags that can be mapped to more than one location on a reference genome, i.e., tags that do not map uniquely, may not be included in the analysis.

The term “site” refers to a unique position (i.e. chromosome ID, chromosome position and orientation) on a reference genome. In some embodiments, a site may provide a position for a residue, a sequence tag, or a segment on a sequence.

As used herein, the terms “aligned,” “alignment,” or “aligning” refer to the process of comparing a read or tag to a reference sequence and thereby determining whether the reference sequence contains the read sequence. If the reference sequence contains the read, the read may be mapped to the reference sequence or, in certain embodiments, to a particular location in the reference sequence. In some cases, alignment simply tells whether or not a read is a member of a particular reference sequence (i.e., whether the read is present or absent in the reference sequence). For example, the alignment of a read to the reference sequence for human chromosome 13 will tell whether the read is present in the reference sequence for chromosome 13. A tool that provides this information may be called a set membership tester. In some cases, an alignment additionally indicates a location in the reference sequence where the read or tag maps to. For example, if the reference sequence is the whole human genome sequence, an alignment may indicate that a read is present on chromosome 13, and may further indicate that the read is on a particular strand and/or site of chromosome 13.

Aligned reads or tags are one or more sequences that are identified as a match in terms of the order of their nucleic acid molecules to a known sequence from a reference genome. Alignment can be done manually, although it is typically implemented by a computer algorithm, as it would be impossible to align reads in a reasonable time period for implementing the methods disclosed herein. One example of an algorithm from aligning sequences is the Efficient Local Alignment of Nucleotide Data (ELAND) computer program distributed as part of the Illumina Genomics Analysis pipeline. Alternatively, a Bloom filter or similar set membership tester may be employed to align reads to reference genomes. See U.S. Patent Application No. 61/552,374 filed Oct. 27, 2011 which is incorporated herein by reference in its entirety. The matching of a sequence read in aligning can be a 100% sequence match or less than 100% (non-perfect match).

The term “mapping” used herein refers to specifically assigning a sequence read to a larger sequence, e.g., a reference genome, by alignment.

The term “derived” when used in the context of a nucleic acid or a mixture of nucleic acids, herein refers to the means whereby the nucleic acid(s) are obtained from the source from which they originate. For example, in one embodiment, a mixture of nucleic acids that is derived from two different genomes means that the nucleic acids, e.g., cfDNA, were naturally released by cells through naturally occurring processes such as necrosis or apoptosis. In another embodiment, a mixture of nucleic acids that is derived from two different genomes means that the nucleic acids were extracted from two different types of cells from a subject.

The term “based on” when used in the context of obtaining a specific quantitative value, herein refers to using another quantity as input to calculate the specific quantitative value as an output.

The term “patient sample” herein refers to a biological sample obtained from a patient, i.e., a recipient of medical attention, care or treatment. The patient sample can be any of the samples described herein. In certain embodiments, the patient sample is obtained by non-invasive procedures, e.g., peripheral blood sample or a stool sample. The methods described herein need not be limited to humans. Thus, various veterinary applications are contemplated in which case the patient sample may be a sample from a non-human mammal (e.g., a feline, a porcine, an equine, a bovine, and the like).

The term “mixed sample” herein refers to a sample containing a mixture of nucleic acids, which are derived from different genomes.

The term “maternal sample” herein refers to a biological sample obtained from a pregnant subject, e.g., a woman.

The term “biological fluid” herein refers to a liquid taken from a biological source and includes, for example, blood, serum, plasma, sputum, lavage fluid, cerebrospinal fluid, urine, semen, sweat, tears, saliva, and the like. As used herein, the terms “blood,” “plasma” and “serum” expressly encompass fractions or processed portions thereof. Similarly, where a sample is taken from a biopsy, swab, smear, etc., the “sample” expressly encompasses a processed fraction or portion derived from the biopsy, swab, smear, etc.

The terms “maternal nucleic acids” and “fetal nucleic acids” herein refer to the nucleic acids of a pregnant female subject and the nucleic acids of the fetus being carried by the pregnant female, respectively.

As used herein, the term “fetal fraction” refers to the fraction of fetal nucleic acids present in a sample comprising fetal and maternal nucleic acid. Fetal fraction is often used to characterize the cfDNA in a mother's blood.

As used herein the term “chromosome” refers to the heredity-bearing gene carrier of a living cell, which is derived from chromatin strands comprising DNA and protein components (especially histones). The conventional internationally recognized individual human genome chromosome numbering system is employed herein.

The term “sensitivity” as used herein refers to the probability that a test result will be positive when the condition of interest is present. It may be calculated as the number of true positives divided by the sum of true positives and false negatives.

The term “specificity” as used herein refers to the probability that a test result will be negative when the condition of interest is absent. It may be calculated as the number of true negatives divided by the sum of true negatives and false positives.

### Introduction and Context

A pregnant mother's blood includes circulating cell-free DNA, some of which originate from the fetus carried by the mother, and some from the mother. For NITP, cfDNA including maternal and fetal DNA may be extracted from the plasma of the peripheral blood of the pregnant mother. The cfDNA may then be used to determine genetic conditions of the fetus, such as copy number variations (CNVs).

Maternal plasma samples represent a mixture of maternal and fetal cfDNA, the fetal cfDNA having a lower fraction than the maternal cfDNA. The success of any given NIPT method for detecting fetal conditions depends on its sensitivity to detect changes in the low fetal fraction samples. For counting based methods, their sensitivity is determined by (a) sequencing depth and (b) ability of data normalization to reduce technical variance. This disclosure provides methods for NIPT and other applications by combining fetal cfDNA and fetal cellular DNA to improve analytical sensitivity of NIPT. Improved analytical sensitivity affords the ability to apply NIPT methods at reduced coverage (e.g., reduced sequencing depth) which enables the use of the technology for lower-cost testing of average risk pregnancies.

Because of the technical difficulties in using cfDNA for NIPT, various techniques and processes have been developed to increase the sensitivity, selectivity or signal-to-noise ratio of cfDNA-based tests. One way to improve the test is to combine information from fetal cfDNA and fetal cellular DNA to improve the test. In an NIPT, the fetal cellular DNA may be obtained from circulating fetal cells (cFCs), which are fetal cells that originate from a fetus and circulate in maternal blood. Example techniques that can be used to obtain fetal cellular DNA from circulating fetal cells are described hereinafter. After fetal cellular DNA is obtained, it can be combined with fetal cfDNA to determine genetic conditions of the fetus. For example, U.S. patent application Ser. No. 14/802,873 describes various techniques to combine fetal cfDNA and fetal cellular DNA to improve the sensitivity, selectivity, or accuracy of NIPT.

Typically, cFCs, such as fetal nucleated red blood cells (fetal NRBCs), exist in maternal blood in very low concentrations. Therefore, fetal cellular DNA obtained from cFCs needs to be combined with fetal cfDNA to provide reliable NIPT test results. As estimated in U.S. Patent Application Publication No. 2013/0122492, there are only about one to two fetal NRBCs in a milliliter of maternal blood. Given the low cFC concentration, it is difficult to obtain or isolate the cFCs from maternal peripheral blood. Sometimes only a single cell or a small number of cells can be isolated from a maternal peripheral blood sample.

To further complicate the matter, unlike fetal cfDNA that quickly clear up in a mother's peripheral blood after a pregnancy, a fetal cell may persist in maternal blood for a long period of time after a pregnancy ends. This means that any fetal cells isolated from a pregnant woman cannot safely be assumed to have originated from the current pregnancy. If the results of prenatal testing are based on a cell originating from a historical pregnancy, this could lead to a serious misdiagnosis.

In contrast to cFCs, fetal cfDNA has a very short plasma half-life and is rapidly cleared from the maternal circulation after the pregnancy is delivered. Therefor cfDNA obtained from a maternal peripheral blood sample can be confidently attributed to either the pregnant mother or the fetus of the ongoing pregnancy.

Some implementations of the disclosure provide a method to determine with high confidence whether a cFC (or fetal cellular DNA) obtained from a pregnant woman's peripheral blood originates from a fetus of a current pregnancy) or a fetus of a historical pregnancy. The method involves comparing genetic information obtained from fetal cellular DNA with genetic information obtained from fetal cfDNA. The method also makes use of maternal DNA (maternal cfDNA or maternal cellular DNA).

Some implementations involve using cfDNA to determine genotypes of the pregnant mother and the current fetus at informative loci, namely those where the mother is homozygous and the fetus is heterozygous. In some implementations, the informative loci include biallelic loci. In some implementations, the informative loci include SNP loci. The methods also involve counting the number of informative loci where both the fetal cfDNA and the fetal cellular DNA are heterozygous and share same alleles. These loci are referred to as shared loci or matched loci, and the genetic markers at these loci are referred to as shared genetic markers or matched genetic markers. The number of shared genetic markers (or shared loci) is provided to a probabilistic model in a Bayesian framework. The model simulates the number of shared genetic markers (or shared loci) as a random sample drawn from a beta-binomial distribution. The model provides as output probabilities of various scenarios of different origins of the fetal cellular DNA. Based on the probabilities, one can determine the origin of the fetal cellular DNA.

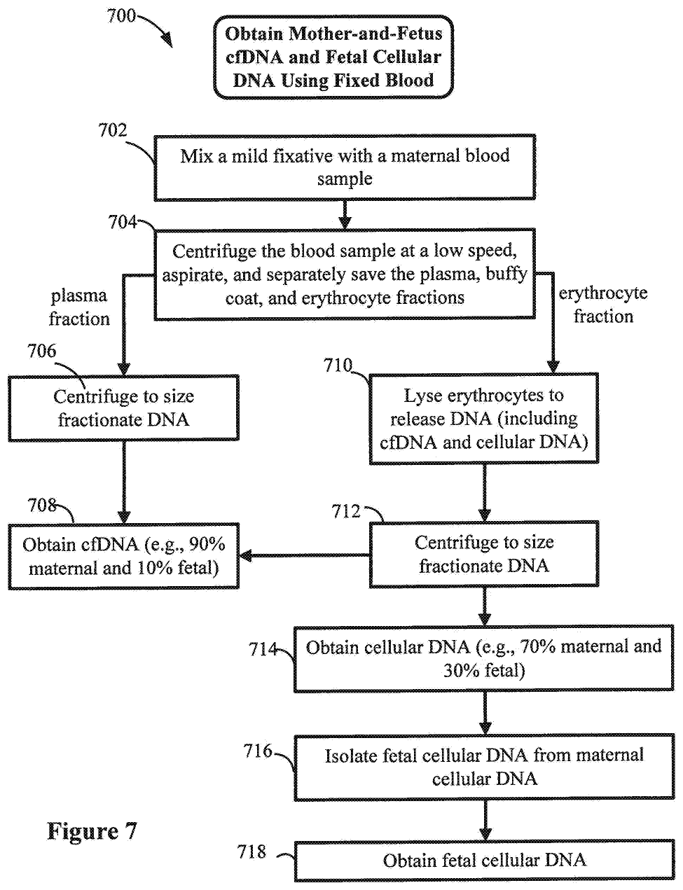

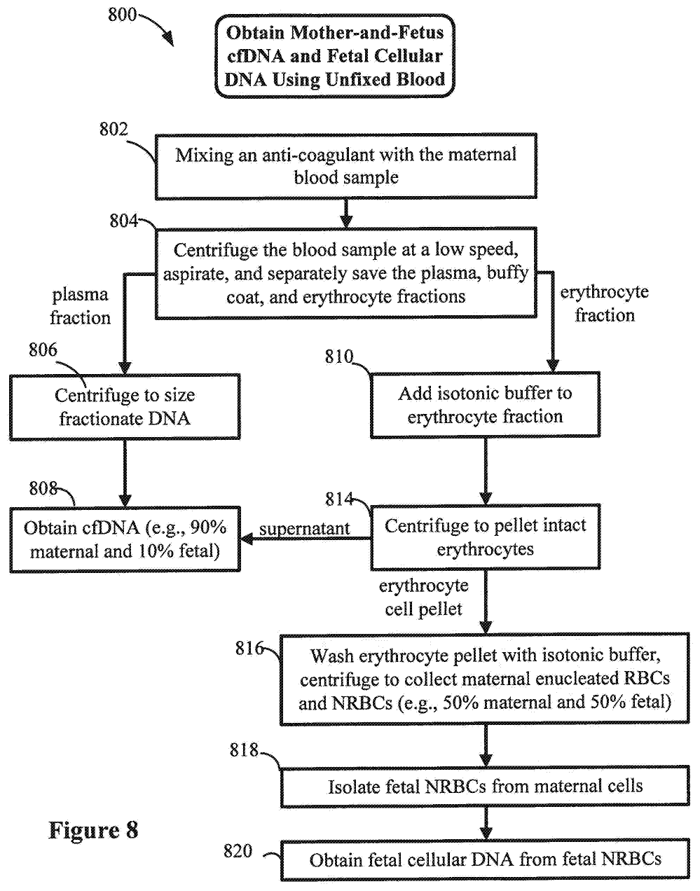

In some implementations, different sources of circulating fetal cells can be determined. In such implementations, identities of the cFCs (in addition to DNA therefrom) are ascertained. Typically for the implementations, the circulating fetal cells are isolated from the maternal sample. This is in contrast to processes where circling fetal cells and circulating maternal cells (e.g., circulating nucleated red blood cells) are processed together, and cellular DNA is obtained from both circling fetal cells and circulating maternal cells. Then fetal cellular DNA can be separated from or identified in the cellular DNA. In the former approach, both the cFCs and the fetal cellular DNA can be identified. See, e.g., FIG. 8. In the latter approach, the fetal cellular DNA (but not the cFCs) can be identified. See, e.g., FIG. 7.

Determining Fetal Conditions Using Fetal Cellular DNA and Fetal cfDNA

Example Workflow for Determining Source of Circulating Fetal Cell

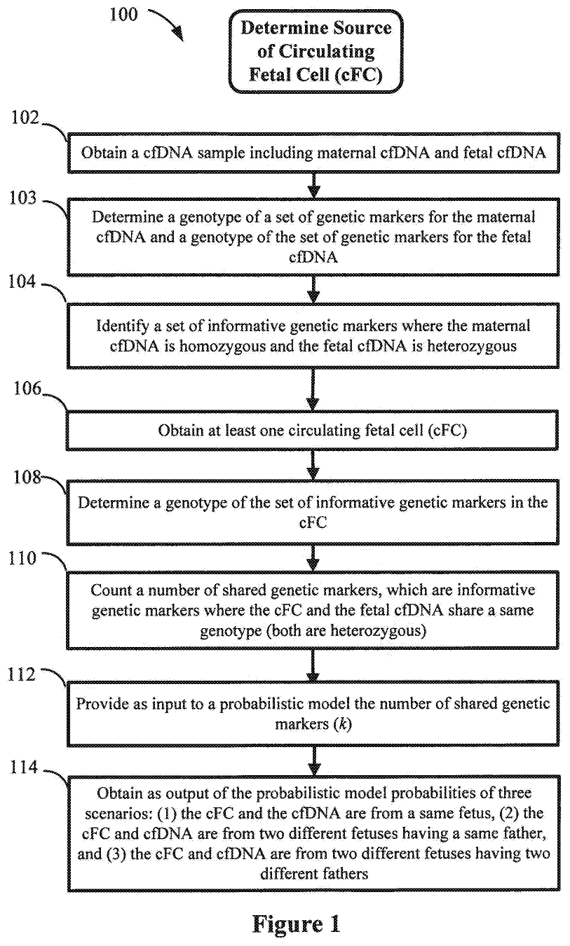

FIG. 1 shows a process 100 for determining different sources of circling fetal cells. Process 100 involves obtaining a cfDNA sample including maternal cfDNA and fetal cfDNA. For instance, a cfDNA sample may be a maternal peripheral blood sample. Other samples may be used as explained hereinafter in the Samples section. Such samples include, but are not limited to sputum/oral fluid, amniotic fluid, blood, a blood fraction, or fine needle biopsy samples (e.g., surgical biopsy, fine needle biopsy, etc.), urine, peritoneal fluid, pleural fluid, and the like.

The methods disclosed herein assume the female carrying the fetus is the genetic mother of a fetus in question, as opposed to a surrogate carrier who does not contribute to half of the fetus's genome. Various techniques may be used to extract cfDNA from a plasma fraction of the maternal peripheral blood sample. Some example techniques for extracting cfDNA are described hereinafter under the Samples section.

Process 100 further involves determining a genotype of a set of genetic markers for the maternal cfDNA and a genotype of the set of genetic markers for the fetal cfDNA. See block 103. A genotype of the set of genetic markers includes alleles at specific genetic loci. In some implementations, the genetic markers include alleles at polymorphic loci. In some implementations, the polymorphic loci are biallelic. Process 100 further involves identifying a set of informative genetic markers (among the set of genetic markers) where the maternal cfDNA is homozygous and the fetal cfDNA is heterozygous. See block 104.

Process 100 also involves obtaining at least one circulating fetal cell (cFC). See block 106. Various methods for obtaining cFCs are further described hereinafter, such as the method depicted in FIG. 8.

Process 100 further involves determining a genotype of the set of informative genetic markers in the cFC. See block 108. Process 100 also involves counting the number of shared genetic markers (k). Shared genetic markers are informative genetic markers where the genotype of the cFC matches the genotype of the fetal cfDNA (both the cFC and the fetal cfDNA are heterozygous). See block 110.

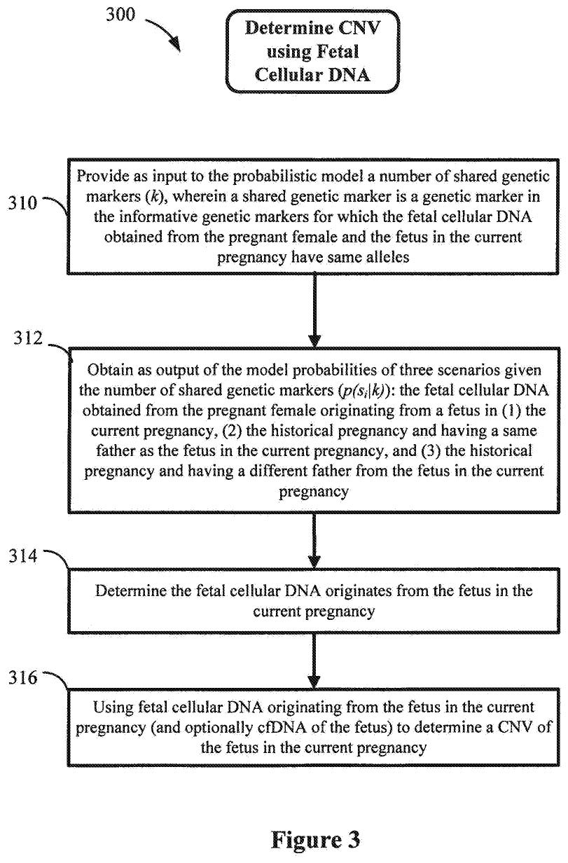

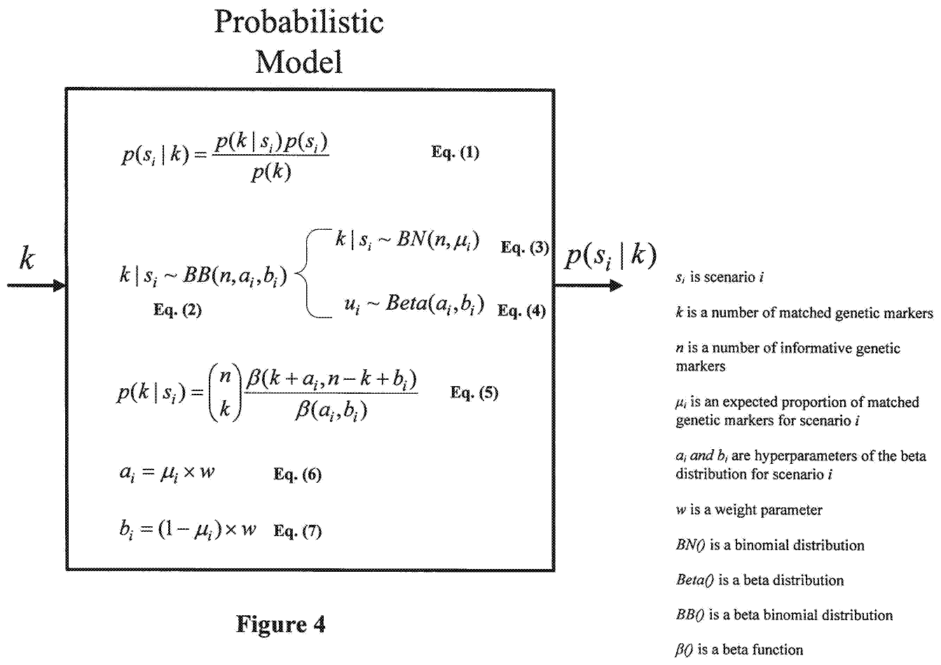

Process 100 further involves providing the number of shared genetic markers (k) to a probabilistic model. See block 112. The probabilistic model may be implemented according to FIGS. 3 and 4. In some implementations, the probabilistic model can be trained using training data and machine learning techniques.

Process 100 then obtains, as output of the probabilistic model, probabilities of three scenarios: (1) the cFC and cfDNA are from the same fetus in the current pregnancy, (2) the cFC in the cfDNA are from two different fetuses having a same father, and (3) the cFC and cfDNA are from two different fetuses having two different fathers. See block 114.

Determining Source of Fetal Cellular DNA

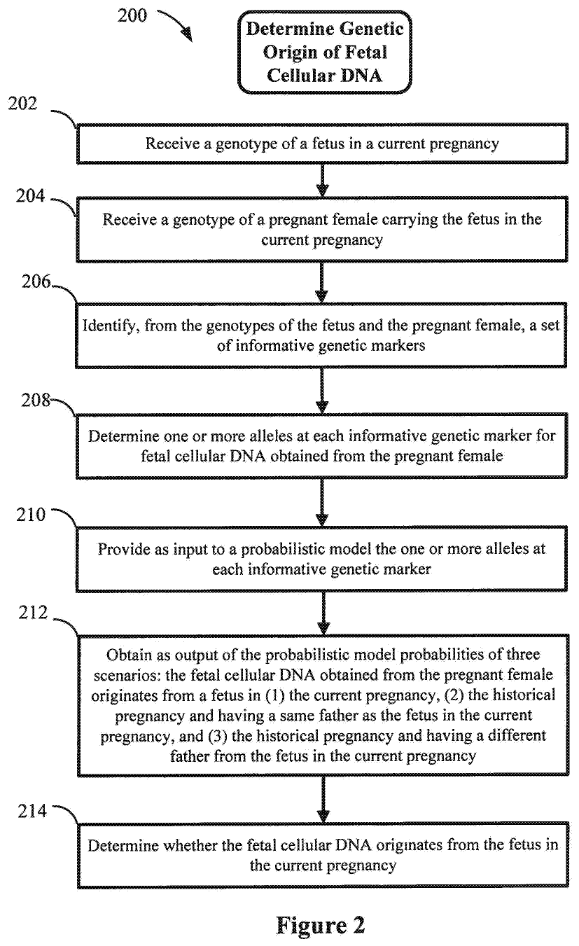

FIG. 2 illustrates a process 200 for determining a genetic origin of fetal cellular DNA or a source of the fetal cellular DNA. The origin or source of the fetal cellular DNA may be a fetus of a current pregnancy or a fetus of a historical pregnancy. For the fetus of a historical pregnancy, it may have a same or different father than the fetus in the current pregnancy. Process 200 is different from process 100 in that the genotype of the fetus in the current pregnancy and the genotype of the pregnant female are not necessarily determined using cfDNA obtained from a maternal blood sample. Moreover, the fetal cellular DNA used in process 200 may be obtained from circulating fetal cells that are either mixed with maternal cells or separated from maternal cells. In contrast, process 100 typically uses circulating fetal cells that have been separated from maternal cells.

Process 200 involves receiving a genotype of a fetus in the current pregnancy. See block 202. In some implementations, the genotype of the fetus in the current pregnancy is obtained from circulating cfDNA that are obtained from a maternal peripheral blood sample. In other implementations, the genotype of the fetus in the current pregnancy may be obtained from other genetic samples, such as sputum/oral fluid, amniotic fluid, blood, a blood fraction, or fine needle biopsy samples (e.g., surgical biopsy, fine needle biopsy, etc.), urine, peritoneal fluid, pleural fluid, and the like. The genotype in this process is defined as one or more alleles at one or more loci in a genome. In some implementations, the one or more loci are polymorphic loci. In some implementations, the polymorphic loci are biallelic loci, where each locus harbors two different alleles.

Process 200 proceeds to receive a genotype of the pregnant female carrying the fetuses. See block 204. In some implementations, the genotype of the pregnant female is obtained from cfDNA extracted from the maternal peripheral blood sample. In some implementations, the cfDNA of the pregnant female and the cfDNA of the fetus are both extracted from the maternal peripheral blood sample. Various techniques may be used to ascertain if a piece of cfDNA comes from the fetus or the mother. In some implementations, the genotype of the pregnant female may be obtained from cellular DNA extracted from maternal cells.

Process 200 further involves identifying, from the genotype of the fetus in the current pregnancy and the genotype of the pregnant female, a set of informative genetic markers. See block 206. Each informative genetic marker is homozygous in the pregnant female and heterozygous in the fetus in the current pregnancy.

Process 200 further involves determining one or more alleles at each informative genetic marker for fetal cellular DNA obtained from the pregnant female. See block 208. The fetal cellular DNA in some implementations is extracted from one or more cFCs found in the blood of the pregnant female. In some implementations, the cFCs have been separated from maternal cells. For example, fetal nucleated red blood cells (nRBCs) are isolated from maternal cells, which isolated fetal nRBCs are used to extract fetal cellular DNA. FIG. 8 illustrates one example process to obtain fetal cellular DNA from fetal NRBCs that have been isolated from maternal cells. In other implementations, cellular DNA of fetal origin and cellular DNA of maternal origin may be obtained from fetal cells and maternal cells that are mixed together. Then the fetal cellular DNA may be separated or isolated from maternal cellular DNA. FIG. 7 illustrates one example process for obtaining fetal cellular DNA by isolating the fetal cellular DNA from maternal cellular DNA.

Process 200 further involves providing as input to the probabilistic model the one or more alleles of each informative genetic markers of the fetal cellular DNA obtained from the pregnant female. See block 210. In some implementations, the one or more alleles at each informative genetic marker of the fetal cellular DNA are compared to one or more alleles at each informative genetic marker of the fetus in the current pregnancy. Then the number of loci (k) where the circulating fetal cellular DNA and the fetus in the current pregnancy share the same two different alleles (the fetus of the current pregnancy is heterozygous at each informative genetic marker) are counted and provided as an input to the probabilistic model. In some implementations, the input to the probabilistic model is implemented as described in blocks 310 in FIG. 3. And the probabilistic model is further described in FIG. 4.

Process 200 also involves obtaining, as output of the probabilistic model, probabilities of three scenarios—the fetal cellular DNA obtained from a pregnant female originates from the fetus (1) in the current pregnancy, (2) in the historic historical pregnancy and having the same father as the fetus in the current pregnancy, and (3) in the historical pregnancy and having a different father from the fetus in the current pregnancy. See block 212.

In some implementations, the model can be extended to cover additional scenarios where the fathers of two fetuses are different but related, such as brothers, cousins, etc. In some implementations, the expected number of shared alleles for different father-father relationships can be modeled by different beta distributions having different parameters. In other implementations, the relationships of different fathers, e.g., brothers, cousins, etc., are modeled by combining mixtures of the two scenarios weighted according to the degree of shared paternal genes, the two scenarios being (a) a historical fetus having the same father as the current fetus and (b) a historical fetus having a father unrelated to the father of the current fetus.

Process 200 then determines whether fetal cellular DNA originates from the fetus in the current pregnancy based on the probability of the three scenarios provided by the model. The scenario having the highest probability is determined as the scenario for the fetal cellular DNA. When the fetal cellular DNA is determined to have originated from the fetus of the current pregnancy, the genetic information of the fetal cellular DNA can be combined with the genetic information of the fetal cfDNA to detect various genetic conditions, such as copy number variation, aneuploidy, and simple nucleotide variation.

FIG. 3 illustrates process 300 for determining copy number variation using fetal cellular DNA originating from a fetus of a current pregnancy and fetal cfDNA from said fetus. Process 300 can use the method described in process 200 to determine that fetal cellular DNA originates from the fetus in the current pregnancy. The process involves providing as input to the probabilistic model a number of shared genetic markers (k). As mentioned above, a shared genetic marker is an informative genetic marker for which the fetal cellular DNA and the fetus in the current pregnancy have same alleles. See block 310. The operation shown in block 310 can be implemented as the operation in block 210 of FIG. 2.

Process 300 further involves obtaining as output of the model probabilities of three scenarios given the number of shared genetic marker markers. The three scenarios are: the fetal cellular DNA obtained from the pregnant female originates from a fetus in (1) a current pregnancy, (2) a historical pregnancy and having the same father as the fetus in the current pregnancy, and (3) the historical pregnancy and having a different father from the fetus in the current pregnancy. See block 312. Process 300 further involves determining that fetal cellular DNA originates from the fetus in the current pregnancy when the probability of scenario (1) is higher than probabilities of the other scenarios. See block 314.

The methods described in process 200 and process 300 do not require direct knowledge of paternal genotypes. The methods can be applied to consanguineous relationships if markers are chosen to avoid regions lacking heterozygosity. In some implementations, the methods can be extended to distinguish between different degrees of relationships between fathers, e.g., brothers, cousins, etc.

Process 300 further involves using fetal cellular DNA originating from the fetus in the current pregnancy to determine a copy number variation of the fetus. In some implementations, genetic information of cfDNA of the fetus is combined with genetic information of the fetal cellular DNA to determine the CNV of the fetus in non-invasive prenatal testing. U.S. patent application Ser. No. 14/802,873 describes various methods to combine genetic information from fetal cellular DNA and genetic information from fetal cfDNA to detect CNV and other genetic conditions. By combining the two types of genetic information, one can improve the sensitivity, selectivity, and signal-to-noise ratio of the NIPT.

FIG. 4 illustrates components of a probabilistic model that can be implemented in process 200 and process 300. The following notations are used to describe the model.

sᵢ is scenario i

k is a number of matched genetic markers

n is a number of informative genetic markers

μᵢ is an expected proportion of matched genetic markers for scenario i

aᵢ and bᵢ are hyperparameters of a beta distribution for scenario i

w is a weight parameter

BN( ) denotes a binomial distribution

Beta( ) denotes a beta distribution

BB( ) denotes a beta binomial distribution

β( ) denotes a beta function

As FIG. 4 illustrates, the probabilistic model takes a number of shared genetic markers (k) as input. A shared genetic marker is a genetic marker in the informative genetic markers for which the fetal cellular DNA obtained from the pregnant female and the fetus in the current pregnancy have the same alleles. The probabilistic model provides as output probabilities of three scenarios given the number of shared genetic markers, p (sᵢ|k). The probabilistic model calculates the probabilities of the three scenarios given the number of shared genetic markers, p(sᵢ|k), based on probabilities of the number of shared genetic markers given the three scenarios, p(k|sᵢ). In some implementations, p(k|sᵢ) is calculated as in equation 1.

$$p ( {s}_{i} \vert k ) = \frac{p ( k \vert {s}_{i} ) p ( {s}_{i} )}{p ( k )} \qquad ( Eq . 1 )$$

Here, p(sᵢ|k) is a probability of scenario i, or sᵢ, given the number of shared genetic markers, or k. p(k|sᵢ) is a probability of the number of shared genetic markers given scenario I. p(sᵢ) is an overall probability of scenario i. p(k) is an overall probability of the number of shared genetic markers.

In some implementations, the probabilistic model simulates the number of shared genetic markers given scenario i, or k|sᵢ, as a random variable drawn from binomial distribution with a success rate μᵢ. In some implementations, k|sᵢ is simulated according to Equation (3).

k|sᵢ˜BN(n,μᵢ) (Eq. 3)

Here, n is a number of informative genetic markers; μᵢ is an expected proportion of matched genetic markers for scenario i.

In some implementation, μᵢ is simulated as a random variable drawn from a beta distribution with hyperparameters of aᵢ and bᵢ. This can be described by Equation 4.

uᵢ˜Beta(aᵢ,bᵢ) (Eq. 4)

Here, aᵢ and bᵢ are hyperparameters of a beta distribution for scenario i.

In these implementations, the probabilistic model simulates, for each scenario, the number of shared genetic markers given scenario i, or k|sᵢ, as a random variable drawn from a beta binomial distribution as illustrated in Equation 2.

k+sᵢ˜BB(n,aᵢ,bᵢ) (Eq. 2)

Here, n is a number of informative genetic markers.

In some implementations, the probability of the number of matched genetic markers k given scenario i is calculated from the following likelihood function in Equation 5.

$$p ( k \vert {s}_{i} ) = ( \begin{matrix}n \\ k\end{matrix} ) \frac{β ( k + {a}_{i} , n - k + {b}_{i} )}{β ( {a}_{i} , {b}_{i} )} \qquad ( Eq . 5 )$$

Here, n is the number of informative genetic markers, k is the number of shared genetic markers, β( ) is a beta function, and aᵢ and bᵢ are the hyperparameters of the beta distribution for scenario i.

In some implementations, the hyperparameter aᵢ is calculated according to Equation 6 and the hyperparameter bᵢ is calculated according to Equation 7.

aᵢ=μᵢ*w (Eq. 6)

bᵢ=(1−μᵢ)*w (Eq. 7)

The parameters aᵢ and bᵢ are calculated from μᵢ, the success rate of the binomial distribution for scenario i, which represents an expected number of shared genetic markers. The weight parameter w can be interpreted as a number of pseudo counts or observations. It determines the concentration of a prior distribution around values corresponding to μ.

In some implementations, the weight parameter w is obtained or refined using a machine learning process. The machine learning process provides a set of training data including three subsets of data obtained from samples under the three different scenarios. The probabilistic model having different values of the weight parameter w is applied to the training data. The weight parameter value providing the best fit to the training data is then used as the weight parameter value to test the genetic origin of cFCs or fetal cellular DNA obtained from the cFCs.

In some implementations, the probabilistic model calculates μ₁, the expected portion of shared genetic markers for scenario (1), according to Equation 8. Scenario (1) is when the fetal cellular DNA obtained from the pregnant female originates from the fetus in the current pregnancy.

$${μ}_{1} = 1 - \frac{1}{n + 1} \qquad ( Eq . 8 )$$

The probabilistic model calculates μ₂, the expected portion of shared genetic markers for scenario (2), according to Equation 9. Scenario (2) is when the fetal cellular DNA obtained from the pregnant female originates from a fetus in a historical pregnancy, and the fetus in the historical pregnancy has a same father as the fetus in the current pregnancy.

$${μ}_{2} = \frac{1}{n} {\sum }_{j = 1}^{n} [ {p}_{j} + \frac{1}{2} ( 1 - {p}_{j} ) ] \qquad ( Eq . 9 )$$

Here, pⱼ is a population frequency of a hetero-allele at the j^{th} marker. The hetero-allele is an allele at an informative genetic marker found in the fetus in the current pregnancy but not in the pregnant female carrying the fetus.

The probabilistic model calculates μ₃, the expected portion of shared genetic markers for scenario (3), according to Equation 10. Scenario (3) is the scenario where the fetal cellular DNA obtained from the pregnant female originates from the fetus in a historical pregnancy, and the fetus in the historical pregnancy has a different father from the fetus in the current pregnancy.

$${μ}_{3} = \frac{1}{n} {\sum }_{j = 1}^{n} {p}_{j} \qquad ( Eq . 10 )$$

In some implementations, prior probabilities of the three scenarios, p(sᵢ), are also provided as input to the model based on known prior information. See Equation (1). The model can take into consideration previously known or expected information relating to the probabilities of the three different scenarios. In some implementations, when a test individual's priors are known, the known prior may be provided to the model. For example, in some implementations, when it is known that the pregnant female likely did not have a previous pregnancy, the probabilities of scenario (2) and (3) may be set to a smaller value. Similarly, the prior probabilities for scenarios (2) and (3) may be set to a particular value if such prior information about previous pregnancies is known. In implementations when factors affecting priors are known for a test individual, such factors may be used to calculate the priors, or priors of a specific population having same factors as the test individual may be used as the test individual's priors.

In some implementations, when a test individual's priors are unknown, default values may be applied based on a general population. In some implementations, when none of the prior pregnancy information is available, some implementations set the probability for the scenarios to be the same.

The probability of observing the number of shared genetic markers, p(k), is a normalizing constant for Equation 1, and can be calculated according to Equation 11.

p(k)=Σᵢp(k|sᵢ)p(sᵢ) (11)

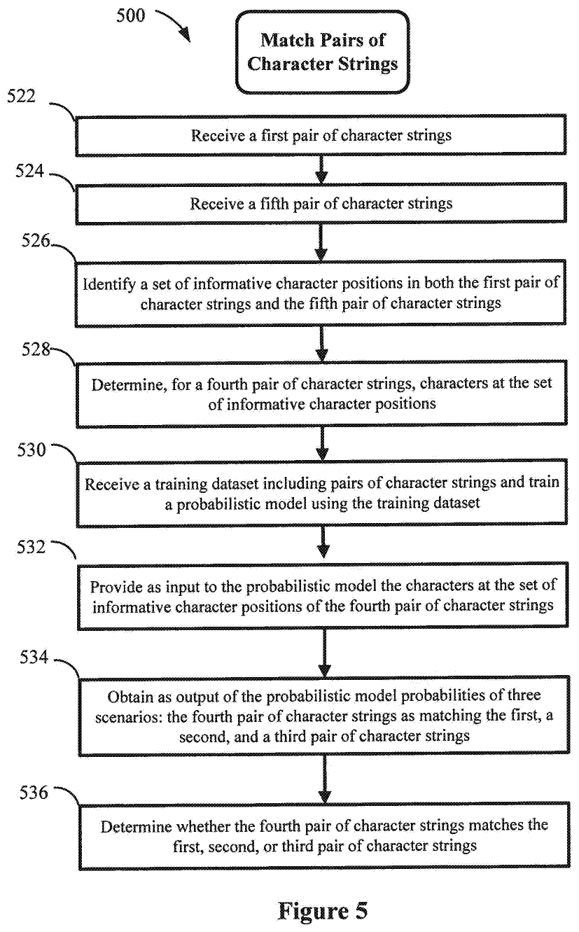

FIG. 5 illustrates process 500 for matching pairs of character strings using probabilistic modeling and computer simulation. The two character strings in any pair have the same number of characters. Some implementations of the method of matching the pairs of character strings can be applied to pairs of genetic sequences or pairs of the genetic marker strings. In some implementations, the character strings comprise different sets of informative genetic markers. Process 500 can be implemented to determine whether one set of genetic markers (e.g., a set of genetic markers of circling fetal cells obtained from a pregnant woman) matches another set of markers (e.g., a set of genetic markers of circling cfDNA of a fetus obtained from the maternal blood sample). Such an implementation corresponds to process 200 illustrated in FIG. 2 and process 300 illustrated in FIG. 3. In some implementations, the character strings comprise sequences of biomolecules, such as polynucleotides, polypeptides, polysaccharides, and other polymers.

Process 500 starts by receiving a first pair of character strings. See block 522. Process 500 also involves receiving a fifth pair of character strings. Two character strings of each pair have the same string size. See block 524. Process 500 further involves identifying a set of informative character positions in both the first pair of character strings and the fifth pair of character strings. See block 526. Each informative character position of the set of informative character positions (a) represents a unique position in each character strings, (b) has one or both of two different characters in any pair of character strings, (c) has only one character of the two different characters in the fifth pair of character strings, and (d) has both characters of the two different characters in the first pair of character strings.

Process 500 further involves determining, for a fourth pair of character strings, characters at the set of informative character positions. See block 528.

Process 500 also involves receiving a training data set including pairs of character strings, and training a probabilistic model using the training data set. See block 530.

Process 500 further involves providing as input to the probabilistic model, characters of the set of informative character positions of the fourth pair of characters strings. See block 532.

Process 500 additionally involves obtaining as output of the probabilistic model probabilities of three scenarios: the fourth pair of character strings matching the first, the second, and the third pair of character strings. See block 534. Each informative character position has a corresponding position on each character strings. The first pair of character strings is attainable by recombining the fifth pair of character strings with a sixth pair of character strings. The second pair of character strings is also obtainable by recombining the fifth pair of character strings with the sixth pair of character strings. The third pair of character strings is obtainable by recombining the fifth pair of character strings with a seventh pair of character strings. Recombining character strings involve using genetic algorithms and techniques reflecting biological recombination of double-stranded DNA, including but not limited to fragmentation, crossover, and mutation.

In some implementations, pairs of character strings correspond to pairs of alleles of a set of genetic markers from parents and offspring. In some implementations, the first pair of character strings corresponds to alleles of a fetus in a current pregnancy for a set of informative genetic markers. The second pair of character strings corresponds to alleles of a fetus in a historical pregnancy that has a same father as the fetus in the current pregnancy. The third pair of character strings corresponds to alleles of a fetus of a historical pregnancy that has a different father than the fetus in the current pregnancy. The fourth pair of character strings corresponds to alleles of fetal cellular DNA obtained from a circulating fetal cell in a maternal blood sample. The fifth pair of character strings corresponds to alleles of the pregnant mother carrying the fetus. The sixth pair of character strings corresponds to alleles of the father of the fetus of the current pregnancy. The seventh pair of character strings corresponds to alleles of a male that is not the father of the fetus of the current pregnancy.

Process 500 further involves determining whether the fourth pair of character strings matches the first, second, or third pair of character strings based on the three probabilities obtained from the probabilistic model. See block 536.

In some implementations, operation 532 includes providing as input to the probabilistic model a number of matched character positions, wherein a matched character position is a character position in the informative character positions for which the fourth pairs of character strings and the first pairs of character strings have same characters. In some implementations, the probabilistic model calculates the probabilities of the three scenarios given the number of matched character positions based on probabilities of the number of matched character position given the three scenarios.

In some implementations, the probabilistic model calculates the probabilities of the three scenarios given a number of matched character positions as

$$p ( {s}_{i} \vert k ) = \frac{p ( k \vert {s}_{i} ) p ( {s}_{i} )}{p ( k )} .$$

Here, p(sᵢ|k) is a probability of scenario i, or sᵢ, given the number of matched character positions, or k. p(k|sᵢ) is a probability of the number of matched character positions given scenario i. p(sᵢ) is an overall probability of scenario i. p(k) is an overall probability of the number of matched character positions.

In some implementations, for each scenario, the probabilistic model simulates the number (k) of matched character positions given scenario i as a random variable drawn from a beta binomial distribution.

In some implementations, the probabilistic model simulates the number of matched character positions given scenario i, or k|sᵢ as a random variable drawn from a binomial distribution with a success rate μᵢ, and μᵢ is a random variable drawn from a beta distribution with hyperparameters aᵢ and bᵢ; namely, k|sᵢ˜BN(n,μᵢ) and μᵢ˜Beta(a_{b}bᵢ), n being the number of informative character positions in the set of informative character positions.

In some implementations, a probability of the number of matched character positions given scenario i is calculated from the following likelihood function:

$$p ( k \vert {s}_{i} ) = ( \begin{matrix}n \\ k\end{matrix} ) \frac{B ( k + {a}_{i} , n - k + {b}_{i} )}{B ( {a}_{i} , {b}_{i} )} .$$

Here, n is the number of informative character positions, k is the number of matched character positions, B( ) is a beta function, and aᵢ and bᵢ are the hyperparameters of the beta distribution for scenario i.

In some implementations, aᵢ=μᵢ*w, and bᵢ=(1−μᵢ)*w, wherein w is a parameter representing a number of pseudo counts or observations. In some implementations, w is obtained from training data using machine learning techniques. The machine learning process provides a set of training data including three subsets of data obtained from samples under the three different scenarios. The probabilistic model having different values of the weight parameter w is applied to the training data. The weight parameter value providing the best fit to the training data is then used as the weight parameter value for w.

Determining CNV Using Fetal Cellular DNA and Fetal cfDNA

This section describes an example workflow for obtaining biological samples from a pregnant mother to extract fetal cellular DNA and fetus-and-mother cfDNA, which are then used to prepare libraries that provide DNA to derive information for determining a sequence of interest for the fetus. In this process, it is important to determine whether the source of the fetal cellular DNA is from a fetus of a current pregnancy or a fetus of a historical pregnancy. After the source of the fetal cellular DNA is determined to be from a fetus of the current pregnancy, information from the cfDNA including DNA of the fetus of the current pregnancy can be combined with information from the cellular DNA of the fetus of the current pregnancy. The combined information can then be used to determine genetic conditions of the fetus. Using the combined information can improve the accuracy, sensitivity, and/or selectivity of diagnoses than using cfDNA alone.

In some embodiments the sequence of interest includes a single nucleotide polymorphism that is related to a medical condition or biological trait. In the embodiments that involve chromosomes or segments of chromosomes, the methods disclosed herein may be used to identify monosomies or trisomies, e.g. trisomy 21 that causes Down Syndrome.

In some embodiments, fetal cellular DNA can be obtained from fetal nucleated red blood cells circulating in the maternal blood, and mother-and-fetus mixed cfDNA can be obtained from the plasma of the maternal blood. The two sources of DNA are then combined and further processed together, in some implementations to obtain two sequencing libraries having indexes identifying the sources of the DNA. If the fetal cellular DNA is from a fetus of the current pregnancy, same as the fetal cfDNA, the sequencing information obtained from the two libraries can be combined to determine a sequence of interest. Some examples below describe how the fetal cfDNA and fetal cellular DNA may be combined to determine the sequence of interest. For instance, in some embodiments, sequence information from the fetal cellular DNA can be used to validate a mosaicism call obtained from cfDNA analysis. Additionally, the combination of sequence information from both the fetal cellular DNA and the cfDNA may provide a higher confidence interval and/or reduce noise in calls for copy number variation, fetal fraction, and/or fetal zygosity. For instance, information from the fetal cellular DNA can be used to reduce the noise in the data, thereby helping to differentiate a homozygous fetus from a heterozygous fetus case (when the mother is heterozygous).

In some embodiments, a targeted amplification and sequencing method can be used. In other embodiments, whole genome amplification may be applied before sequencing. To reduce processing biases and otherwise permit reliable comparison of the cell free nucleic acid sequences and the cellular nucleic acid sequences, the two nucleic acid samples are processed similarly in some embodiments. For example, they can be sequenced in a mixture of the nucleic acids from both samples by a multiplexing technique. In some embodiments, cellular nucleic acids and cell free nucleic acids are obtained from the same sample but then separated and indexed (or otherwise uniquely identified) in the separated fractions and then the fractions are pooled for amplification, sequencing, and the like. In some implementations, the fetal cellular nucleic acid fraction is enhanced before being combined with mother-and-fetus cell free nucleic acid fraction, such that the separately indexed cellular nucleic acid and cell free nucleic acid are made similar with regard to size and concentration prior to pooling for sequencing and other downstream processing.

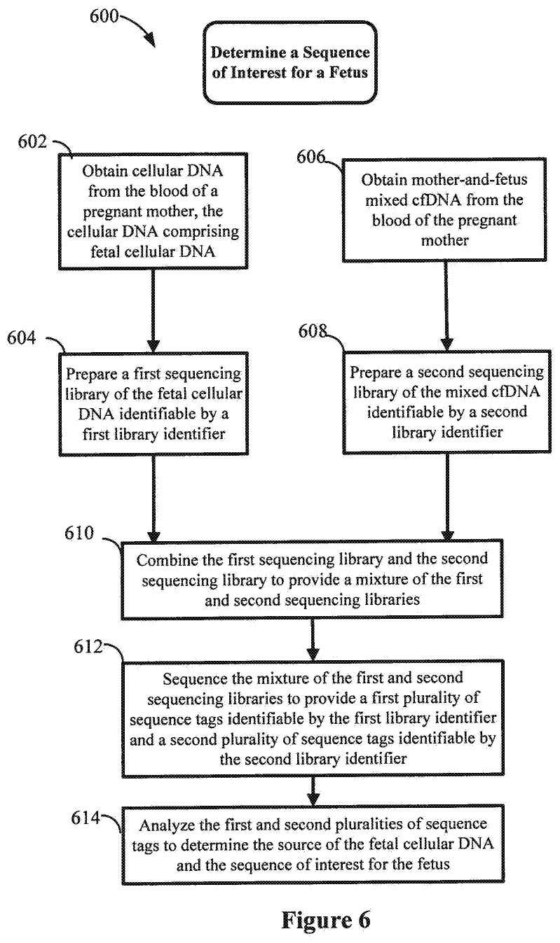

FIG. 6 shows a process flow of a method 600 for determining a sequence of interest of a fetus according to some embodiments of the disclosure. FIGS. 7-9 are specific implementations of various components of the process flow depicted in FIG. 6. In some embodiments, method 600 involves obtaining cellular DNA from a maternal blood sample of a pregnant mother. See block 602. In some embodiments, the cellular DNA includes both maternal cellular DNA and fetal cellular DNA. In some embodiments, the fetal cellular DNA is isolated from maternal cellular DNA before further downstream processing. The fetal cellular DNA includes at least a sequence that maps to the sequence of interest. In some embodiments, the sequence of interest includes polymorphic sequences of a disease related gene. In some embodiments, the sequence of interest comprises a site of an allele associated with a disease. In some embodiments, the sequence of interest comprises one or more of the following: single nucleotide polymorphism, tandem repeat, deletion, insertion, a chromosome or a segment of a chromosome.

In some embodiments, fetal cellular DNA is obtained from fetal nucleated red blood cells (NRBCs) circulating in the maternal blood sample. The fetal cellular DNA and the fetal NRBCs may be obtained from maternal peripheral blood as described herein. In some embodiments, the fetal NRBCs are obtained from an erythrocyte fraction of a maternal blood sample. In some embodiments, the fetal cellular DNA may be obtained from other fetal cell types circulating in the maternal blood.

In some embodiments, the method also involves obtaining mother-and-fetus mixed cfDNA from the pregnant mother. See block 606. The cfDNA includes at least one sequence that maps to the at least one sequence of interest. In some embodiments, the cfDNA is obtained from the plasma of a blood sample from the mother. In some embodiments, the same blood sample also provides the fetal NRBC as the source of the fetal cellular DNA. Of course, the cellular DNA and cfDNA may also be obtained from different samples of the same mother.

In some embodiments, the method applies an indicator of the source of DNA as being from the fetal cellular DNA or from the cfDNA. In some embodiments, this indicator comprises a first library identifier and a second library identifier. In some embodiments, the process involves preparing a first sequencing library of fetal cellular DNA obtained from operation 602, wherein the first sequencing library is identifiable by a first library identifier. Block 604. In some embodiments, the first library identifier is a first index sequence that is identifiable in downstream sequencing steps. In some embodiments, the indicator of the source of DNA also comprises a second sequencing library of the cfDNA identifiable by a second library identifier. Block 608. In preparing sequence libraries, the method may involve incorporating indexes to each of said sequence libraries, wherein the indexes incorporated to said first library differ from the indexes incorporated to said second library. The indexes contain unique sequences (e.g., bar codes) that are identifiable in downstream sequencing steps, thereby providing an indicator of the source of the nucleic acids.

In some embodiments, the indicator of the source of DNA may be provided by other methods such as size separation.

In some embodiments, the method proceeds by combining at least a portion of the fetal cellular DNA of the first sequencing library and at least a portion of the cfDNA of the second sequencing library to provide a mixture of the first and second sequencing libraries. See block 610.

In FIG. 6, preparation of the first sequencing library and the second sequencing library is shown as two separate branches of the workflow, and the prepared libraries are combined to obtain a mixture of the first and second sequencing libraries. However, in some embodiments the two libraries are indexed separately at the beginning, then further processed in a combined sample. In some embodiments, the method involves further processing the combined sample to prepare or modify sequencing libraries. In some embodiments, the further processing involves incorporating sequencing adaptors (e.g., paired end primers) for massively parallel sequencing.

In some embodiments, the method then proceeds with sequencing at least a portion of the mixture of the first and second sequencing libraries to provide a first plurality of sequence tags identifiable by the first library identifier and a second plurality of sequence tags identifiable by second library identifier. See block 612. In some embodiments, the sequence reads are then mapped to a reference sequence containing the sequence of interest, thereby providing sequence tags mapped to the sequence of interest. In some embodiments, the sequence of interest may identify the presence of an allele. In some embodiments, the sample has been selectively enriched for the sequence of interest.

In some embodiments, instead of or in addition to selective enrichment of the sequence of interest before sequencing, the sample may be amplified by whole genome amplification. In some of these embodiments, the sequence reads are aligned to a reference genome comprising a sequence of interest (e.g., chromosome, chromosome segment) that are typically longer than in the embodiment with selective enrichment targeting shorter sequences of interest (e.g., SNPs, STRs, and sequences of up to kb in size). The sequence reads mapping to the sequence of interest provide sequence tags for the sequence of interest, which can be used to determine a genetic condition, e.g., aneuploidy, related to the sequence of interest.

In some embodiments, the method applies massively parallel sequencing. Various sequencing techniques may be used, including but not limited to, sequencing by synthesis and sequencing by ligation. In some embodiments, sequencing by synthesis uses reversible dye terminators. In some embodiments, single molecule sequencing is used.

In some embodiments, the method further involves analyzing the first and second pluralities of sequence tags to determine the at least one sequence of interest. See block 614. At least a portion of the plurality of sequence tags map to the at least one sequence of interest. In some embodiments, the method determines the presence or abundance of sequence tags mapping to the sequence of interest. This may include determining CNV (e.g., aneuploidy) and non-NCV abnormality. Particularly, the method may determine the relative amounts of two alleles in each of the cfDNA and cellular DNA. In some embodiments, the method may detect that the fetus has a genetic disorder by determining that the fetus is homozygous of a disease causing allele of a disease related gene wherein the mother is heterozygous of the allele.

In some embodiments, the method starts with cellular DNA and cfDNA in separate reaction environments, e.g., test tubes. In some embodiments, the method involves enriching wild-type and mutant regions using probes that target both alleles of disease related gene(s) and have different indices for cellular DNA and cfDNA, the indices are incorporated into the targeted sequences in the separate reaction environment. The method further involves mixing the cellular DNA and cfDNA with enriched targeted regions and amplifying the DNA using universal PCR primers. In some embodiments, whole genome amplification instead of targeted sequence amplification is applied. The amplified product will be sequencing-ready libraries of both cellular DNA of the fetus and cfDNA for the mother and fetus. The sequencing results may then be used to determine a sequence of interest for the fetus. In some embodiments, determining the sequence of interest provides information for detecting a CNV or non-CNV chromosomal anomaly involving the sequence of interest. In some embodiments, the method may determine the zygosity of the fetus and/or fetal fraction of the cfDNA.

In some embodiments, the method further involves determining a plurality of training sequences from the cfDNA and the cellular DNA, which can be used to determine a CNV or non-CNV chromosomal anomaly involving a sequence of interest. Some embodiments further use the sequence information obtained from the cellular DNA to determine the fetal fraction of the cfDNA. The methods exemplified in FIG. 6 and set forth above with respect to DNA can be carried out for other nucleic acids (e.g. mRNA) as well.

Obtaining cfDNA and Fetal Cellular DNA

In various embodiments, mother-and-fetus mixed cfDNA and fetal cellular DNA are obtained from maternal peripheral blood to provide the genetic materials, as respectively shown in block 602 and block 606 of FIG. 6. The genetic materials are used to generate two identifiable libraries as respectively shown in block 604 and block 608 of FIG. 6. The two libraries are then combined for further downstream processing and analyses. Various methods may be used to obtain cfDNA and fetal cellular DNA. Two processes are described below as examples to illustrate applicable methods for obtaining cfDNA and fetal cellular DNA for downstream processing and analyses.

A Process of Obtaining DNA Using Fixed Blood

Fetal cellular DNA and mixed cfDNA may be obtained from fixed or unfixed blood samples. Maternal peripheral blood samples can be collected using any of a number of various different techniques. Techniques suitable for individual sample types will be readily apparent to those of skill in the art. For example, in certain embodiments, blood is collected in specially designed blood collection tubes or other container. Such tubes may include an anti-coagulant such as ethylenediamine tetracetic acid (EDTA) or acid citrate dextrose (ACD). In some cases, the tube includes a fixative. In some embodiments, blood is collected in a tube that gently fixes cells and deactivates nucleases (e.g., Streck Cell-free DNA BCT tubes). See US Patent Application Publication No. 2010/0209930, filed Feb. 11, 2010, and US Patent Application Publication No. 2010/0184069, filed Jan. 19, 2010 each previously incorporated herein by reference.

FIG. 7 depicts a flowchart of a process 700 to obtain mother-and-fetus cfDNA and fetal cellular DNA using a fixed whole blood sample obtained from a pregnant mother. Of course, the process may be modified to use two samples from the same pregnant mother, with one sample providing cfDNA and one providing cellular DNA. Process 700 begins with mixing a mild fixative with a maternal blood sample that includes cellular DNA and cfDNA. Block 702. The cellular DNA may originate from maternal cells and/or fetal cells. The blood sample can be collected by any one of many available techniques. Such techniques should collect a sufficient volume of sample to supply enough cfDNA to satisfy the requirements of the sequencing technology, and account for losses during the processing leading up to sequencing.

In certain embodiments, blood is collected in specially designed blood collection tubes or other container. Such tubes may include an anti-coagulant such as ethylenediamine tetracetic acid (EDTA) or acid citrate dextrose (ACD). In some cases, the tube includes a fixative. In some embodiments, blood is collected in a tube that gently fixes cells and deactivates nucleases (e.g., Streck Cell-free DNA BCT tubes). See US Patent Application Publication No. 2010/0209930, filed Feb. 11, 2010, and US Patent Application Publication No. 2010/0184069, filed Jan. 19, 2010 each previously incorporated herein by reference.

Generally, it is desirable to collect and process cfDNA that is uncontaminated with DNA from other sources such as white blood cells. Therefore, white blood cells can be removed from the sample and/or treated in a manner that reduces the likelihood that they will release their DNA.

Process 700 then proceed to separate a plasma fraction from an erythrocyte fraction of the fixed blood sample. In some embodiments, to separate the plasma fraction from the erythrocyte fraction, the process centrifuges the blood sample at a low speed, then aspirates and separately saves the plasma, buffy coat, and erythrocyte fractions. See block 704.

In some embodiments, the blood sample is centrifuged, sometimes for multiple times. The first centrifugation step applies a low speed to produce three fractions: a plasma fraction on top, a buffy coat containing leukocytes, and an erythrocyte fraction on the bottom. This first centrifugation process is performed at relatively low g-force in order to avoid disrupting the hematocytes (e.g. leukocytes, nucleated erythrocytes, and platelets) to a point where their nuclei break apart and release DNA into the plasma fraction. Density gradient centrifugation is typically used. If this first centrifugation step is performed at too high of an acceleration, some DNA from the leukocytes would likely contaminate the plasma fraction. After this centrifugation step is completed, the plasma fraction and erythrocyte fraction are separated from each other and can be further processed.

The plasma fraction can be subjected to a second higher speed centrifugation to size fractionate DNA, removing larger particulates from the plasma, leaving cfDNA in the plasma. See block 706. In this step, additional particulate matter from the plasma is pelleted as a solid phase and removed. This additional solid material may include some additional cells that also contain DNA that would otherwise contaminate the cell free DNA that is to be analyzed. In some embodiments, the first centrifugation is performed at an acceleration of about 1600 g and the second centrifugation is performed at an acceleration of about 16,000 g.

While a single centrifugation process from normal blood is possible to obtain cfDNA, such process has been found to sometimes produce plasma contaminated with white blood cells. Any DNA isolated from this plasma will include some cellular DNA. Therefore, for cfDNA isolation from normal blood, the plasma may be subjected to a second centrifugation at high-speed to pellet out any contaminating cells.

After removing larger sized particulates from the plasma by size fractionation, the process 700 proceeds to isolate/purify cfDNA from the plasma. See block 708. In some embodiments, the isolation can be performed by the following operations.

A. Denature and/or degrade proteins in plasma (e.g. contact with proteases) and add guanidine hydrochloride or other chaotropic reagent to the solution (to facilitate driving cfDNA out of solution)

B. Contact treated plasma with a support matrix such as beads in a column. cfDNA comes out of solution and binds to matrix.

C. Wash the support matrix.

D. Release cfDNA from matrix and recover the cfDNA for downstream process (e.g., indexed library preparation) and statistical analyses.

After a plasma fraction is collected as described, the cfDNA is extracted. Extraction is actually a multistep process that involves separating DNA from the plasma in a column or other solid phase binding matrix. The extracted cfDNA usually includes both maternal and fetal cfDNA. Depending on the pregnancy stage and physiological condition of the mother and the fetus, the cfDNA can include up to 10% of fetal DNA in some examples.

The first part of this cfDNA isolation procedure involves denaturing or degrading the nucleosome proteins and otherwise taking steps to free the DNA from the nucleosome. A typical reagent mixture used to accomplish this isolation includes a detergent, protease, and a chaotropic agent such as guanine hydrochloride. The protease serves to degrade the nucleosome proteins, as well as background proteins in the plasma such as albumin and immunoglobulins. The chaotropic agent disrupts the structure of macromolecules by interfering with intramolecular interactions mediated by non-covalent forces such as hydrogen bonds. The chaotropic agent also renders components of the plasma such as proteins negative in charge. The negative charge makes the medium somewhat energetically incompatible with the negatively charged DNA. The use of a chaotropic agent to facilitate DNA purification is described in Boom et al., “Rapid and Simple Method for Purification of Nucleic Acids”, J. Clin. Microbiology, v. 28, No. 3, 1990.

After this protein degradation treatment, which frees, at least partially, the DNA coils from the nucleosome proteins, the resulting solution is passed through a column or otherwise exposed to support matrix. The cfDNA in the treated plasma selectively adheres to the support matrix. The remaining constituents of the plasma pass through the binding matrix and are removed. The negative charge imparted to medium components facilitates adsorption of DNA in the pores of a support matrix.

After passing the treated plasma through the support matrix, the support matrix with bound cfDNA is washed to remove additional proteins and other unwanted components of the sample. After washing, the cfDNA is freed from the matrix and recovered. Notably, this process loses a significant fraction of the available DNA from the plasma. Generally, support matrixes have a high capacity for cfDNA, which limits the amount of cfDNA that can be easily separated from the matrix. As a consequence, the yield of cfDNA extraction step can be quite low. Typically, the efficiency is well below 50% (e.g., it has been found that the typical yield of cfDNA is 4-12 ng/ml of plasma from the available ˜30 ng/ml plasma).

Other methods may be used to obtain cfDNA from a maternal blood sample with higher yield. One example is further described here. For instance, in one embodiment, a device can be used to collect 2-4 drops of patient blood (100-200 ul) and then separate the plasma from the hematocrit using a specialized membrane. The device can be used to generate the required 50-100 μl of plasma for NGS library preparation. Once the plasma has been separated by the membrane, it can be absorbed into a pretreated medical sponge. In certain embodiments, the sponge is pretreated with a combination of preservatives, proteases and salts to (a) inhibit nucleases and/or (b) stabilize the plasma DNA until downstream processing. Products such as Vivid Plasma Separation Membrane (Pall Life Sciences, Ann Arbor, Mich.) and Medisponge 50PW (Filtrona technologies, St. Charles, Mich.) can be used. The plasma DNA in the medical sponge can be accessed for NGS library generation in a variety of ways. (a) Reconstitute and extract that plasma from the sponge and isolate DNA for downstream processing. Of course, this approach may have limited DNA recovery efficiency. (b) Utilize the DNA-binding properties of the medical sponge polymer to isolate the DNA. (c) Conduct direct PCR-based library preparation using the DNA that is bound to the sponge. This may be conducted using any of the cfDNA library preparation techniques described herein.

The purified cfDNA obtained from operation 708 can be used to prepare a library for sequencing. To sequence a population of double-stranded DNA fragments using massively parallel sequencing systems, the DNA fragments must be flanked by known adapter sequences. A collection of such DNA fragments with adapters at either end is called a sequencing library. Two examples of suitable methods for generating sequencing libraries from purified DNA are (1) ligation-based attachment of known adapters to either end of fragmented DNA, and (2) transposase-mediated insertion of adapter sequences. There are many suitable massively parallel sequencing techniques. Some of these are described below.

Note that operations 702-708 described so far for process 700 depicted in FIG. 7 largely overlap with operations 802-808 in process 800 of FIG. 8 described below.

Process 700 also provides fetal cellular DNA from the maternal blood sample, which makes use of the erythrocyte fraction obtained from the low-speed centrifugation of operation 704. In some embodiments, the process involves lysing the erythrocytes in the erythrocyte fraction DNA, the product from which includes both cfDNA and cellular DNA. See block 710. Next, process 700 proceeds by centrifuging the sample to size fractionate DNA, allowing the separation of cfDNA and cellular DNA, since cfDNA is much smaller in size than cellular DNA as described above. See block 712. In some embodiments, this centrifugation operation may be similar to the centrifugation of operation 706, performed at 16,000 g. In some implementations, the cfDNA obtained from the erythrocyte fraction may optionally be combined with the cfDNA obtained from the plasma fraction for downstream processing. See block 708.

Process 700 allows obtaining cellular DNA from the erythrocyte fraction. See block 714. The cellular DNA obtained from the erythrocytes fraction largely originates from NRBCs. During pregnancy, most of the NRBC that are present in the maternal blood stream are those that have been produced by the mother herself. See Wachtel, et al., Prenat. Diagn. 18: 455-463 (1998). In some instances, the cellular DNA include up to 50% of fetal cellular DNA. For example, the cellular DNA may include 70% of maternal DNA and 30% of fetal DNA as shown by Wachtel et al.

In some embodiments, process 700 proceeds by isolating the fetal cellular DNA from maternal cellular DNA. See block 706. Various methods may be applied to separate the two sources of cellular DNA by taking advantage of the different characteristics of the two sources of DNA. See block 716. For instance, it has been shown that fetal DNA tends to have a higher state of methylation than maternal DNA. Therefore, mechanisms that differentiate methylation may be used to separate fetal cellular DNA from maternal cellular DNA. See, e.g., Kim et al., Am J Reprod Immunol. 2012 July; 68(1):8-27, for different methylation characteristics of maternal versus fetal cells.

Additionally, FISH can be used to detect and localize specific DNA or RNA targets from fetal cells. Some embodiments may ascertain fetal origin by FISH that identifies fetal specific DNA markers. Therefore, process 700 allows one to obtain fetal cellular DNA, which can then be further processed and analyzed. See block 718.

A Process of Obtaining DNA Using Unfixed Blood

The disclosure also provides methods for obtaining fetal cellular DNA and mixed cfDNA using unfixed blood samples. FIG. 8 is a flowchart showing a process of such a method. The operations for obtaining cfDNA depicted in FIG. 8 largely overlap with those in the process depicted in FIG. 7. Therefore blocks 704, 706 and 708 mirror blocks 804, 806 and 808.

Briefly, process 800 starts by mixing an anti-coagulant such as EDTA or ACD with the maternal blood sample without using a fixative. See block 802. Process 800 proceeds by separating a plasma fraction and an erythrocyte fraction from the blood sample by centrifugation. See block 804. As in block 804, the centrifugation may be performed at a lower-speed, such as 1600 g. The sample is then aspirated, and plasma, buffy coat, and the erythrocyte fractions are separately saved. The plasma fraction obtained from operation 804 and then undergo a second centrifugation at a higher speed such as 16,000 g to size fractionate DNA, spinning out larger particulates and leaving smaller cfDNA in the plasma. See block 806. Process 800 provides means to obtain cfDNA from the plasma that can be used for further processing and analysis. See block 808.

Operations 810-818 of process 800 allow isolation of fetal NRBCs from the erythrocyte fraction, and obtaining fetal cellular DNA from the isolated fetal NRBCs. Operation 810 involves adding isotonic buffer to the erythrocyte fraction. Then the process proceeds by centrifugation to pellet intact erythrocytes. See block 814. In some embodiments, this centrifugation is performed at a lower speed than that in operation 806 in order to avoid rupturing the erythrocytes. The supernatant from this centrifugation includes cfDNA that can be combined with the cfDNA obtained from the plasma fraction for downstream processing and analysis. See block 808. The pellet, or compacted precipitant, includes intact erythrocytes from both the mother and the fetus, wherein the erythrocytes from the mother include a large portion of enucleated RBCs and a small number of NRBCs.

In some embodiments, process 800 proceeds by washing erythrocyte pellet with isotonic buffer, then centrifuging to collect maternal enucleated RBCs and NRBCs. The NRBCs include both maternal and fetal NRBCs, with up to 30% of fetal cells in some embodiments as discussed above. Process 800 then proceeds by isolating fetal NRBCs from maternal cells. See block 818. One can then obtain fetal cellular DNA from the isolated fetal NRBCs. See block 820.

Isolate Fetal NRBC and Fetal Cellular DNA

In various embodiments, such as operations 818 and 820 of process 800 depicted in FIG. 8, fetal NRBCs are isolated from maternal cells, and fetal cellular DNA is obtained from the isolated fetal NRBCs. Various combinations of methods may be applied to isolate NRBCs from maternal cells. In some embodiments, the methods can include various combinations of cell sorting with magnetic particles or flow cytometry, density gradient centrifugation, size-based separation, selective cell lysis, or depletion of unwanted cell populations. Often, these methods alone are not effective because each method may be able to remove some unwanted cells but not all. Therefore combination of methods can be used to isolate the desired fetal NRBCs.

In some embodiments, isolation of fetal NRBCs is combined with enrichment of the fetal NRBCs by one or more methods known in the art or described herein. The enrichment increases the concentration of rare cells or ratio of rare cells to non-rare cells in the sample. In some embodiments, when enriching fetal cells from a maternal peripheral venous blood sample, the initial concentration of the fetal cells may be about 1:50,000,000 and it may be increased to at least 1:5,000 or 1:500. Enrichment can be achieved by one or more types of separation modules described herein or in the prior art. See, e.g., U.S. Pat. No. 8,137,912 for some techniques for enrichment of fetal cells, which is incorporated by reference in its entirety. Multiple separation modules may be coupled in series for enhanced performance.

In some embodiments, the fetal cellular DNA used for downstream processing is obtained from one or more fetal NRBCs in the blood of the pregnant mother. In some embodiments, the method separates the fetal NRBCs from maternal erythrocytes in a cellular component of a blood sample of the pregnant mother. In some embodiments, separating the fetal NRBCs from the maternal erythrocytes comprises differentially lysing maternal erythrocytes. In some embodiments, separating the fetal NRBCs from the maternal erythrocytes comprises size-based separation and/or capture-based separation. The capture-based separation may comprise capturing the fetal NRBCs through binding one or more cellular markers expressed by fetal NRBCs. Preferably, the one or more cellular markers comprise a surface marker expressed by fetal NRBCs but not, or to a lesser degree, by maternal NRBCs. In some embodiments, the capture-based separation comprise binding magnetically responsive particles to fetal NRBCs, wherein the magnetically responsive particles have an affinity to one or more cellular markers expressed by fetal NRBCs. In some embodiments, the capture-based separation is performed by an automated immunomagnetic separation device, for example, as described in U.S. Pat. No. 8,071,395, which is incorporated herein by reference. In some embodiments, the capture-based separation comprises binding fluorescent labels to fetal NRBCs, wherein the fluorescent labels have an affinity to one or more cellular markers expressed by fetal NRBCs.

In various embodiments, cell surface markers expressed on fetal NRBCs are used for affinity based separation. For instance, some embodiments may use anti-CD71 to attach magnetic or fluorescent probes to transferrin receptors, which probes provide a mechanism for magnetic-activated cell sorting (MACS) or fluorescence-activated cell sorting (FACS). Cells from very early developmental stages can be isolated from umbilical cord blood using CD34. To enrich and identify erythroid cells from later developmental stages, surface markers such as CD71, glycophorin A, CD36, antigen-i, and intracellularly expressed hemoglobins may be used. Soybean agglutinin (SBA) may be used to isolate fetal NRBCs from the blood of pregnant mothers.

Many of the above surface markers are not exclusive to fetal NRBCs. Instead, they are also expressed to various degrees on maternal cells. Recently, monoclonal anti-bodies have been identified with affinity to fetal NRBCs but not to maternal bloods. For instance, Zimmermann et al. identified monoclonal antibody clones 4B8 and 4B9 that has specific affinity to fetal NRBCs. Experimental Cell Research, 319 (2013), 2700-2707. The mAb 4B8, 4B9 and other similar mABs may be used to provide binding mechanism for MACS or FACS to isolate fetal NRBCs. Magnetism based cell separation may be implemented as a MagSweeper device, which is an automated immunomagnetic separation technology as disclosed in U.S. Pat. No. 8,071,395, which is incorporated by reference in its entirety. In some implementations, the MagSweeper can enrich circulating rare cells, e.g., fetal NRBCs in maternal blood, by an order of 10⁸-fold increase in concentration.

The fetal origin of isolated cells can be indicated by PCR amplification of Y chromosome specific sequences, by fluorescence in situ hybridization (FISH), by detecting ε-globin and γ-globin, or by comparing DNA-polymorphisms with STR-markers from mother and child. Some embodiments may use these indicators to separate fetal NRBCs from other cells, e.g., implemented as imaging-based separation mechanism by visualizing the indicator or as affinity-based separation mechanism by hybridizing with the indicator.

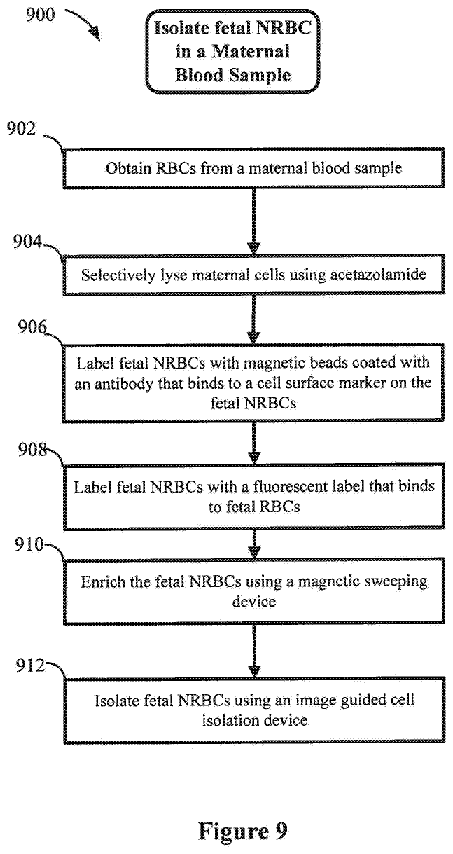

FIG. 9 is a flowchart showing process 900 for isolating fetal NRBCs from a maternal blood sample according to some embodiments of the disclosure. Process 900 relates to process 800 in that process 900 provides one example of how operation 818 in FIG. 8 may be accomplished. Process 900 starts by obtaining RBCs from maternal blood sample, see block 902, such as using one or more density gradient centrifugations as described in the steps leading to step 816.

The process then proceeds to remove maternal enucleated RBCs and NRBCs from the RBCs by selectively lysing maternal erythrocytes using acetazolamide and lysing solutions containing NH₄⁺ and HCO₃⁺. See block 904. Erythrocytes can be quickly disrupted in lysing solutions containing NH₄⁺ and HCO₃⁺, Carbonic anhydrase catalyzes this hemolysis reaction, and is at least 5-fold lower in fetal cells than adult cells. Therefore the hemolytic rate is slower for fetal cells. This differential of hemolysis is augmented by acetazolamide, which is an inhibitor of carbonic anhydrase, and which penetrates fetal cell about 10 times faster than adult cells. Therefore the combination of acetazolamide and lysing solutions containing NH₄⁺ and HCO₃⁺ selectively lyses the maternal cells while sparing the fetal cells.

In one embodiment, the differential lyses may be performed as in the following example. The RBCs are centrifuged (e.g., 300 g, 10 min), re-suspended in phosphate-buffered saline (PBS) with acetazolamide, and incubated at room temperature for 5 min. Two and one half milliliters of lysis buffer (10 mM NaHCO₃, 155 mM NH₄Cl) is added and the cells are incubated for 5 min, centrifuged, re-suspended in lysis buffer, incubated for 3 min, and centrifuged.

After the selectively lysing maternal RBCs, lysed cells may be removed by centrifugation. In some embodiments, the process proceeds to label fetal NRBCs with magnetic beads coated with an antibody that binds to a cell surface marker expressed on the fetal NRBCs. See block 906. One or more of the surface markers expressed on fetal NRBCs described above may be the target for binding. In some embodiments, mAb 4B8, mAb 4B9, or anti-CD71 may be used as the antibody that binds to the surface of fetal NRBCs. The magnetic beads provides a means for magnetic separation mechanism to capture the fetal NRBCs, which are then selectively enriched. In some embodiments, the process proceeds to label the fetal NRBCs with a fluorescent label, e.g., oligonucleotides (“oligos”) bound to fluorescein or rhodamine, which oligos bind to mRNA of markers of fetal NRBCs. In some embodiments, the fluorescent label binds to the mRNA of fetal hemoglobin, e.g., ε-globin and γ-globin.

Process 900 proceeds to enrich the fetal NRBCs using magnetic separation device such as the MagSweeper described above, which captures the NRBCs through the magnetic beads selectively attached to the NRBCs. See block 910. Finally, process 900 achieves isolation of fetal NRBCs using an image guided cell isolation device such as a FACS sensitive to the fluorescent label attached to the fetal NRBCs in operation 908. See block 912. The isolated fetal NRBCs may then be used to prepare an indexed fetal cellular DNA library. Some embodiments of the preparation of the indexed library are further described below.

In many embodiments, fetal NRBCs are first isolated from maternal RBCs and other cell types. Then fetal cellular DNA is obtained from the isolated fetal NRBCs. However, in some embodiments, fetal cellular DNA may be obtained by selectively lysing fetal NRBCs (as opposed to lysing the maternal cells). For example, fetal cells can be selectively lysed releasing their nuclei when a blood sample including fetal cells is combined with deionized water. Such selective lysis of the fetal cells allows for the subsequent enrichment of fetal DNA using, e.g., size or affinity based separation.

### Samples

Samples used herein contain nucleic acids that are “cell-free” (e.g., cfDNA) or cell-bound (e.g., cellular DNA). Cell-free nucleic acids, including cell-free DNA, can be obtained by various methods known in the art from biological samples including but not limited to plasma, serum, and urine (see, e.g., Fan et al., Proc Natl Acad Sci 105:16266-16271 [2008]; Koide et al., Prenatal Diagnosis 25:604-607 [2005]; Chen et al., Nature Med. 2: 1033-1035 [1996]; Lo et al., Lancet 350: 485-487 [1997]; Botezatu et al., Clin Chem. 46: 1078-1084, 2000; and Su et al., J Mol. Diagn. 6: 101-107 [2004]). To separate cell-free DNA from cells in a sample, various methods including, but not limited to fractionation, centrifugation (e.g., density gradient centrifugation), DNA-specific precipitation, or high-throughput cell sorting and/or other separation methods can be used. Commercially available kits for manual and automated separation of cfDNA are available (Roche Diagnostics, Indianapolis, Ind., Qiagen, Valencia, Calif., Macherey-Nagel, Duren, Del.). Biological samples comprising cfDNA have been used in assays to determine the presence or absence of chromosomal abnormalities, e.g., trisomy 21, by sequencing assays that can detect chromosomal aneuploidies and/or various polymorphisms.

In various embodiments the DNA present in the sample can be enriched specifically or non-specifically prior to use (e.g., prior to preparing a sequencing library). Non-specific enrichment of sample DNA refers to the whole genome amplification of the genomic DNA fragments of the sample that can be used to increase the level of the sample DNA prior to preparing a DNA sequencing library. Non-specific enrichment can be the selective enrichment of one of the two genomes present in a sample that comprises more than one genome. For example, non-specific enrichment can be selective of the cancer genome in a plasma sample, which can be obtained by known methods to increase the relative proportion of cancer to normal DNA in a sample. Alternatively, non-specific enrichment can be the non-selective amplification of both genomes present in the sample. For example, non-specific amplification can be of cancer and normal DNA in a sample comprising a mixture of DNA from the cancer and normal genomes. Methods for whole genome amplification are known in the art. Degenerate oligonucleotide-primed PCR (DOP), primer extension PCR technique (PEP) and multiple displacement amplification (MDA) are examples of whole genome amplification methods. In some embodiments, the sample comprising the mixture of cfDNA from different genomes is un-enriched for cfDNA of the genomes present in the mixture. In other embodiments, the sample comprising the mixture of cfDNA from different genomes is non-specifically enriched for any one of the genomes present in the sample.

The sample comprising the nucleic acid(s) to which the methods described herein are applied typically comprises a biological sample (“test sample”), e.g., as described above. In some embodiments, the nucleic acid(s) to be analyzed is purified or isolated by any of a number of well-known methods.

Accordingly, in certain embodiments the sample comprises or consists of a purified or isolated polynucleotide, or it can comprise samples such as a tissue sample, a biological fluid sample, a cell sample, and the like. Suitable biological fluid samples include, but are not limited to blood, plasma, serum, sweat, tears, sputum, urine, sputum, ear flow, lymph, saliva, cerebrospinal fluid, ravages, bone marrow suspension, vaginal flow, trans-cervical lavage, brain fluid, ascites, milk, secretions of the respiratory, intestinal and genitourinary tracts, amniotic fluid, milk, and leukophoresis samples. In some embodiments, the sample is a sample that is easily obtainable by non-invasive procedures, e.g., blood, plasma, serum, sweat, tears, sputum, urine, sputum, ear flow, saliva or feces. In certain embodiments the sample is a peripheral blood sample, or the plasma and/or serum fractions of a peripheral blood sample. In other embodiments, the biological sample is a swab or smear, a biopsy specimen, or a cell culture. In another embodiment, the sample is a mixture of two or more biological samples, e.g., a biological sample can comprise two or more of a biological fluid sample, a tissue sample, and a cell culture sample. As used herein, the terms “blood,” “plasma” and “serum” expressly encompass fractions or processed portions thereof. Similarly, where a sample is taken from a biopsy, swab, smear, etc., the “sample” expressly encompasses a processed fraction or portion derived from the biopsy, swab, smear, etc.

In certain embodiments, samples can be obtained from sources, including, but not limited to, samples from different individuals, samples from different developmental stages of the same or different individuals, samples from different diseased individuals (e.g., individuals with cancer or suspected of having a genetic disorder), normal individuals, samples obtained at different stages of a disease in an individual, samples obtained from an individual subjected to different treatments for a disease, samples from individuals subjected to different environmental factors, samples from individuals with predisposition to a pathology, samples individuals with exposure to an infectious disease agent (e.g., HIV), and the like.

The sample used in the disclosure processes can be a tissue sample, a biological fluid sample, or a cell sample. A biological fluid includes, as non-limiting examples, blood, plasma, serum, sweat, tears, sputum, urine, sputum, ear flow, lymph, saliva, cerebrospinal fluid, ravages, bone marrow suspension, vaginal flow, transcervical lavage, brain fluid, ascites, milk, secretions of the respiratory, intestinal and genitourinary tracts, and leukophoresis samples.

In another illustrative, but non-limiting embodiment, the donee sample is a mixture of two or more biological samples, e.g., the biological sample can comprise two or more of a biological fluid sample, a tissue sample, and a cell culture sample. In some embodiments, the sample is a sample that is easily obtainable by non-invasive procedures, e.g., blood, plasma, serum, sweat, tears, sputum, urine, milk, sputum, ear flow, saliva and feces. In some embodiments, the biological sample is a peripheral blood sample, and/or the plasma and serum fractions thereof. In other embodiments, the biological sample is a swab or smear, a biopsy specimen, or a sample of a cell culture. As disclosed above, the terms “blood,” “plasma” and “serum” expressly encompass fractions or processed portions thereof. Similarly, where a sample is taken from a biopsy, swab, smear, etc., the “sample” expressly encompasses a processed fraction or portion derived from the biopsy, swab, smear, etc.

In certain embodiments samples can also be obtained from in vitro cultured tissues, cells, or other polynucleotide-containing sources. The cultured samples can be taken from sources including, but not limited to, cultures (e.g., tissue or cells) maintained in different media and conditions (e.g., pH, pressure, or temperature), cultures (e.g., tissue or cells) maintained for different periods of length, cultures (e.g., tissue or cells) treated with different factors or reagents (e.g., a drug candidate, or a modulator), or cultures of different types of tissue and/or cells.

Methods of isolating nucleic acids from biological sources are well known and will differ depending upon the nature of the source. One of skill in the art can readily isolate nucleic acid(s) from a source as needed for the method described herein. In some instances, it can be advantageous to fragment the nucleic acid molecules in the nucleic acid sample. Fragmentation can be random, or it can be specific, as achieved, for example, using restriction endonuclease digestion. Methods for random fragmentation are well known in the art, and include, for example, limited DNAse digestion, alkali treatment and physical shearing. In one embodiment, sample nucleic acids are obtained from as cfDNA, which is not subjected to fragmentation.

### Sequencing Library Preparation

In one embodiment, the methods described herein can utilize next generation sequencing technologies (NGS), that allow multiple samples to be sequenced individually as genomic molecules (i.e., singleplex sequencing) or as pooled samples comprising indexed genomic molecules (e.g., multiplex sequencing) on a single sequencing run. These methods can generate up to several hundred million reads of DNA sequences. In various embodiments the sequences of genomic nucleic acids, and/or of indexed genomic nucleic acids can be determined using, for example, the Next Generation Sequencing Technologies (NGS) described herein. In various embodiments analysis of the massive amount of sequence data obtained using NGS can be performed using one or more processors as described herein.

In various embodiments the use of such sequencing technologies does not involve the preparation of sequencing libraries.

However, in certain embodiments the sequencing methods contemplated herein involve the preparation of sequencing libraries. In one illustrative approach, sequencing library preparation involves the production of a random collection of adapter-modified DNA fragments (e.g., polynucleotides) that are ready to be sequenced. Sequencing libraries of polynucleotides can be prepared from DNA or RNA, including equivalents, analogs of either DNA or cDNA, for example, DNA or cDNA that is complementary or copy DNA produced from an RNA template, by the action of reverse transcriptase. The polynucleotides may originate in double-stranded form (e.g., dsDNA such as genomic DNA fragments, cDNA, PCR amplification products, and the like) or, in certain embodiments, the polynucleotides may originated in single-stranded form (e.g., ssDNA, RNA, etc.) and have been converted to dsDNA form. By way of illustration, in certain embodiments, single stranded mRNA molecules may be copied into double-stranded cDNAs suitable for use in preparing a sequencing library. The precise sequence of the primary polynucleotide molecules is generally not material to the method of library preparation, and may be known or unknown. In one embodiment, the polynucleotide molecules are DNA molecules. More particularly, in certain embodiments, the polynucleotide molecules represent the entire genetic complement of an organism or substantially the entire genetic complement of an organism, and are genomic DNA molecules (e.g., cellular DNA, cell free DNA (cfDNA), etc.), that typically include both intron sequence and exon sequence (coding sequence), as well as non-coding regulatory sequences such as promoter and enhancer sequences. In certain embodiments, the primary polynucleotide molecules comprise human genomic DNA molecules, e.g., cfDNA molecules present in peripheral blood of a pregnant subject.

Preparation of sequencing libraries for some NGS sequencing platforms is facilitated by the use of polynucleotides comprising a specific range of fragment sizes. Preparation of such libraries typically involves the fragmentation of large polynucleotides (e.g. cellular genomic DNA) to obtain polynucleotides in the desired size range.

Fragmentation can be achieved by any of a number of methods known to those of skill in the art. For example, fragmentation can be achieved by mechanical means including, but not limited to nebulization, sonication and hydroshear. However mechanical fragmentation typically cleaves the DNA backbone at C—O, P—O and C—C bonds resulting in a heterogeneous mix of blunt and 3′- and 5′-overhanging ends with broken C—O, P—O and/C—C bonds (see, e.g., Alnemri and Liwack, J Biol. Chem 265:17323-17333 [1990]; Richards and Boyer, J Mol Biol 11:327-240 [1965]) which may need to be repaired as they may lack the requisite 5′-phosphate for the subsequent enzymatic reactions, e.g., ligation of sequencing adaptors, that are required for preparing DNA for sequencing.

In contrast, cfDNA, typically exists as fragments of less than about 300 base pairs and consequently, fragmentation is not typically necessary for generating a sequencing library using cfDNA samples.

Typically, whether polynucleotides are forcibly fragmented (e.g., fragmented in vitro), or naturally exist as fragments, they are converted to blunt-ended DNA having 5′-phosphates and 3′-hydroxyl. Standard protocols, e.g., protocols for sequencing using, for example, the Illumina platform as described elsewhere herein, instruct users to end-repair sample DNA, to purify the end-repaired products prior to dA-tailing, and to purify the dA-tailing products prior to the adaptor-ligating steps of the library preparation.

Various embodiments of methods of sequence library preparation described herein obviate the need to perform one or more of the steps typically mandated by standard protocols to obtain a modified DNA product that can be sequenced by NGS. An abbreviated method (ABB method), a 1-step method, and a 2-step method are examples of methods for preparation of a sequencing library, which can be found in patent application Ser. No. 13/555,037 filed on Jul. 20, 2012, which is incorporated by reference by its entirety.

### Sequencing Methods

As indicated above, the prepared samples (e.g., Sequencing Libraries) are sequenced as part of the disclosed procedures. Any of a number of sequencing technologies can be utilized.

Some sequencing technologies are available commercially, such as the sequencing-by-hybridization platform from Affymetrix Inc. (Sunnyvale, Calif.) and the sequencing-by-synthesis platforms from 454 Life Sciences (Bradford, Conn.), Illumina/Solexa (Hayward, Calif.) and Helicos Biosciences (Cambridge, Mass.), and the sequencing-by-ligation platform from Applied Biosystems (Foster City, Calif.), as described below. In addition to the single molecule sequencing performed using sequencing-by-synthesis of Helicos Biosciences, other single molecule sequencing technologies include, but are not limited to, the SMRT™ technology of Pacific Biosciences, the ION TORRENT™ technology, and nanopore sequencing developed for example, by Oxford Nanopore Technologies.

While the automated Sanger method is considered as a “first generation” technology, Sanger sequencing including the automated Sanger sequencing, can also be employed in the methods described herein. Additional suitable sequencing methods include, but are not limited to nucleic acid imaging technologies, e.g., atomic force microscopy (AFM) or transmission electron microscopy (TEM). Illustrative sequencing technologies are described in greater detail below.

In one illustrative, but non-limiting, embodiment, the methods described herein comprise obtaining sequence information for the nucleic acids in a test sample, e.g., cfDNA or cellular DNA sample in a subject being screened for a genetic disorder, a cancer, and the like, using Illumina's sequencing-by-synthesis and reversible terminator-based sequencing chemistry (e.g. as described in Bentley et al., Nature 6:53-59 [2009]). Template DNA can be genomic DNA, e.g., cellular DNA or cfDNA. In some embodiments, genomic DNA from isolated cells is used as the template, and it is fragmented into lengths of several hundred base pairs. In other embodiments, cfDNA is used as the template, and fragmentation is not required as cfDNA exists as short fragments. For example fetal cfDNA circulates in the bloodstream as fragments approximately 170 base pairs (bp) in length (Fan et al., Clin Chem 56:1279-1286 [2010]), and no fragmentation of the DNA is required prior to sequencing. Circulating tumor DNA also exist in short fragments, with a size distribution peaking at about 150-170 bp. Illumina's sequencing technology relies on the attachment of fragmented genomic DNA to a planar, optically transparent surface on which oligonucleotide anchors are bound. Template DNA is end-repaired to generate 5′-phosphorylated blunt ends, and the polymerase activity of Klenow fragment is used to add a single A base to the 3′ end of the blunt phosphorylated DNA fragments. This addition prepares the DNA fragments for ligation to oligonucleotide adapters, which have an overhang of a single T base at their 3′ end to increase ligation efficiency. The adapter oligonucleotides are complementary to the flow-cell anchor oligos (not to be confused with the anchor/anchored reads in the analysis of repeat expansion). Under limiting-dilution conditions, adapter-modified, single-stranded template DNA is added to the flow cell and immobilized by hybridization to the anchor oligos. Attached DNA fragments are extended and bridge amplified to create an ultra-high density sequencing flow cell with hundreds of millions of clusters, each containing about 1,000 copies of the same template. In one embodiment, the randomly fragmented genomic DNA is amplified using PCR before it is subjected to cluster amplification. Alternatively, an amplification-free (e.g., PCR free) genomic library preparation is used, and the randomly fragmented genomic DNA is enriched using the cluster amplification alone (Kozarewa et al., Nature Methods 6:291-295 [2009]). The templates are sequenced using a robust four-color DNA sequencing-by-synthesis technology that employs reversible terminators with removable fluorescent dyes. High-sensitivity fluorescence detection is achieved using laser excitation and total internal reflection optics. Short sequence reads of about tens to a few hundred base pairs are aligned against a reference genome and unique mapping of the short sequence reads to the reference genome are identified using specially developed data analysis pipeline software. After completion of the first read, the templates can be regenerated in situ to enable a second read from the opposite end of the fragments. Thus, either single-end or paired end sequencing of the DNA fragments can be used.

Various embodiments of the disclosure may use sequencing by synthesis that allows paired end sequencing. In some embodiments, the sequencing by synthesis platform by Illumina involves clustering fragments. Clustering is a process in which each fragment molecule is isothermally amplified. In some embodiments, as the example described here, the fragment has two different adaptors attached to the two ends of the fragment, the adaptors allowing the fragment to hybridize with the two different oligos on the surface of a flow cell lane. The fragment further includes or is connected to two index sequences at two ends of the fragment, which index sequences provide labels to identify different samples in multiplex sequencing. In some sequencing platforms, a fragment to be sequenced is also referred to as an insert.

In some implementation, a flow cell for clustering in the Illumina platform is a glass slide with lanes. Each lane is a glass channel coated with a lawn of two types of oligos. Hybridization is enabled by the first of the two types of oligos on the surface. This oligo is complementary to a first adapter on one end of the fragment. A polymerase creates a compliment strand of the hybridized fragment. The double-stranded molecule is denatured, and the original template strand is washed away. The remaining strand, in parallel with many other remaining strands, is clonally amplified through bridge application.

In bridge amplification, a strand folds over, and a second adapter region on a second end of the strand hybridizes with the second type of oligos on the flow cell surface. A polymerase generates a complimentary strand, forming a double-stranded bridge molecule. This double-stranded molecule is denatured resulting in two single-stranded molecules tethered to the flow cell through two different oligos. The process is then repeated over and over, and occurs simultaneously for millions of clusters resulting in clonal amplification of all the fragments. After bridge amplification, the reverse strands are cleaved and washed off, leaving only the forward strands. The 3′ ends are blocked to prevent unwanted priming.

After clustering, sequencing starts with extending a first sequencing primer to generate the first read. With each cycle, fluorescently tagged nucleotides compete for addition to the growing chain. Only one is incorporated based on the sequence of the template. After the addition of each nucleotide, the cluster is excited by a light source, and a characteristic fluorescent signal is emitted. The number of cycles determines the length of the read. The emission wavelength and the signal intensity determine the base call. For a given cluster all identical strands are read simultaneously. Hundreds of millions of clusters are sequenced in a massively parallel manner. At the completion of the first read, the read product is washed away.

In the next step of protocols involving two index primers, an index 1 primer is introduced and hybridized to an index 1 region on the template. Index regions provide identification of fragments, which is useful for de-multiplexing samples in a multiplex sequencing process. The index 1 read is generated similar to the first read. After completion of the index 1 read, the read product is washed away and the 3′ end of the strand is de-protected. The template strand then folds over and binds to a second oligo on the flow cell. An index 2 sequence is read in the same manner as index 1. Then an index 2 read product is washed off at the completion of the step.

After reading two indices, read 2 initiates by using polymerases to extend the second flow cell oligos, forming a double-stranded bridge. This double-stranded DNA is denatured, and the 3′ end is blocked. The original forward strand is cleaved off and washed away, leaving the reverse strand. Read 2 begins with the introduction of a read 2 sequencing primer. As with read 1, the sequencing steps are repeated until the desired length is achieved. The read 2 product is washed away. This entire process generates millions of reads, representing all the fragments. Sequences from pooled sample libraries are separated based on the unique indices introduced during sample preparation. For each sample, reads of similar stretches of base calls are locally clustered. Forward and reversed reads are paired creating contiguous sequences. These contiguous sequences are aligned to the reference genome for variant identification.

The sequencing by synthesis example described above involves paired end reads, which is used in many of the embodiments of the disclosed methods. Paired end sequencing involves two reads from the two ends of a fragment. When a pair of reads are mapped to a reference sequence, the base-pair distance between the two reads can be determined, which distance can then be used to determine the length of the fragments from which the reads were obtained. In some instances, a fragment straddling two bins would have one of its pair-end read aligned to one bin, and another to an adjacent bin. This gets rarer as the bins get longer or the reads get shorter. Various methods may be used to account for the bin-membership of these fragments. For instance, they can be omitted in determining fragment size frequency of a bin; they can be counted for both of the adjacent bins; they can be assigned to the bin that encompasses the larger number of base pairs of the two bins; or they can be assigned to both bins with a weight related to portion of base pairs in each bin.

Paired end reads may use insert of different length (i.e., different fragment size to be sequenced). As the default meaning in this disclosure, paired end reads are used to refer to reads obtained from various insert lengths. In some instances, to distinguish short-insert paired end reads from long-inserts paired end reads, the latter is also referred to as mate pair reads. In some embodiments involving mate pair reads, two biotin junction adaptors first are attached to two ends of a relatively long insert (e.g., several kb). The biotin junction adaptors then link the two ends of the insert to form a circularized molecule. A sub-fragment encompassing the biotin junction adaptors can then be obtained by further fragmenting the circularized molecule. The sub-fragment including the two ends of the original fragment in opposite sequence order can then be sequenced by the same procedure as for short-insert paired end sequencing described above. Further details of mate pair sequencing using an Illumina platform is shown in an online publication at the following URL, which is incorporated by reference by its entirety: res|.|illumina|.|com/documents/products/technotes/technote_nextera_matepair_data_processing. Additional information about paired end sequencing can be found in U.S. Pat. No. 7,601,499 and US Patent Publication No. 2012/0,053,063, which are incorporated by reference with regard to materials on paired end sequencing methods and apparatuses.

After sequencing of DNA fragments, sequence reads of predetermined length, e.g., 100 bp, are mapped or aligned to a known reference genome. The mapped or aligned reads and their corresponding locations on the reference sequence are also referred to as tags. In one embodiment, the reference genome sequence is the NCBI36/hg18 sequence, which is available on the world wide web at genome|.|ucsc|.|edu/cgi-bin/hgGateway?org=Human&db=hg18&hgsid=166260105). Alternatively, the reference genome sequence is the GRCh37/hg19, which is available on the World Wide Web at genome dot ucsc dot edu/cgi-bin/hgGateway. Other sources of public sequence information include GenBank, dbEST, dbSTS, EMBL (the European Molecular Biology Laboratory), and the DDBJ (the DNA Databank of Japan). A number of computer algorithms are available for aligning sequences, including without limitation BLAST (Altschul et al., 1990), BLITZ (MPsrch) (Sturrock & Collins, 1993), FASTA (Person & Lipman, 1988), BOWTIE (Langmead et al., Genome Biology 10:R25.1-R25.10 [2009]), or ELAND (Illumina, Inc., San Diego, Calif., USA). In one embodiment, one end of the clonally expanded copies of the plasma cfDNA molecules is sequenced and processed by bioinformatics alignment analysis for the Illumina Genome Analyzer, which uses the Efficient Large-Scale Alignment of Nucleotide Databases (ELAND) software.

In one illustrative, but non-limiting, embodiment, the methods described herein comprise obtaining sequence information for the nucleic acids in a test sample using single molecule sequencing technology of the Helicos True Single Molecule Sequencing (tSMS) technology (e.g. as described in Harris T. D. et al., Science 320:106-109 [2008]). In the tSMS technique, a DNA sample is cleaved into strands of approximately 100 to 200 nucleotides, and a polyA sequence is added to the 3′ end of each DNA strand. Each strand is labeled by the addition of a fluorescently labeled adenosine nucleotide. The DNA strands are then hybridized to a flow cell, which contains millions of oligo-T capture sites that are immobilized to the flow cell surface. In certain embodiments the templates can be at a density of about 100 million templates/cm2. The flow cell is then loaded into an instrument, e.g., HeliScope™ sequencer, and a laser illuminates the surface of the flow cell, revealing the position of each template. A CCD camera can map the position of the templates on the flow cell surface. The template fluorescent label is then cleaved and washed away. The sequencing reaction begins by introducing a DNA polymerase and a fluorescently labeled nucleotide. The oligo-T nucleic acid serves as a primer. The polymerase incorporates the labeled nucleotides to the primer in a template directed manner. The polymerase and unincorporated nucleotides are removed. The templates that have directed incorporation of the fluorescently labeled nucleotide are discerned by imaging the flow cell surface. After imaging, a cleavage step removes the fluorescent label, and the process is repeated with other fluorescently labeled nucleotides until the desired read length is achieved. Sequence information is collected with each nucleotide addition step. Whole genome sequencing by single molecule sequencing technologies excludes or typically obviates PCR-based amplification in the preparation of the sequencing libraries, and the methods allow for direct measurement of the sample, rather than measurement of copies of that sample.

### Apparatus and System for Determining Sources of Fetal Cellular DNA

Analysis of the sequencing data and the diagnosis derived therefrom are typically performed using various computer executed algorithms and programs. Therefore, certain embodiments employ processes involving data stored in or transferred through one or more computer systems or other processing systems. Embodiments disclosed herein also relate to apparatus for performing these operations. This apparatus may be specially constructed for the required purposes, or it may be a general-purpose computer (or a group of computers) selectively activated or reconfigured by a computer program and/or data structure stored in the computer. In some embodiments, a group of processors performs some or all of the recited analytical operations collaboratively (e.g., via a network or cloud computing) and/or in parallel. A processor or group of processors for performing the methods described herein may be of various types including microcontrollers and microprocessors such as programmable devices (e.g., CPLDs and FPGAs) and non-programmable devices such as gate array ASICs or general purpose microprocessors.

In addition, certain embodiments relate to tangible and/or non-transitory computer readable media or computer program products that include program instructions and/or data (including data structures) for performing various computer-implemented operations. Examples of computer-readable media include, but are not limited to, semiconductor memory devices, magnetic media such as disk drives, magnetic tape, optical media such as CDs, magneto-optical media, and hardware devices that are specially configured to store and perform program instructions, such as read-only memory devices (ROM) and random access memory (RAM). The computer readable media may be directly controlled by an end user or the media may be indirectly controlled by the end user. Examples of directly controlled media include the media located at a user facility and/or media that are not shared with other entities. Examples of indirectly controlled media include media that is indirectly accessible to the user via an external network and/or via a service providing shared resources such as the “cloud.” Examples of program instructions include both machine code, such as produced by a compiler, and files containing higher level code that may be executed by the computer using an interpreter.

In various embodiments, the data or information employed in the disclosed methods and apparatus is provided in an electronic format. Such data or information may include reads and tags derived from a nucleic acid sample, counts or densities of such tags that align with particular regions of a reference sequence (e.g., that align to a chromosome or chromosome segment), reference sequences (including reference sequences providing solely or primarily polymorphisms), calls such as SNV or aneuploidy calls, counseling recommendations, diagnoses, and the like. As used herein, data or other information provided in electronic format is available for storage on a machine and transmission between machines. Conventionally, data in electronic format is provided digitally and may be stored as bits and/or bytes in various data structures, lists, databases, etc. The data may be embodied electronically, optically, etc.

One embodiment provides a computer program product for determining sources of fetal cellular DNA and/or using the fetal cellular DNA to determine fetal genetic conditions. The computer product may contain instructions for performing any one or more of the above-described methods for determining a chromosomal anomaly. As explained, the computer product may include a non-transitory and/or tangible computer readable medium having a computer executable or compilable logic (e.g., instructions) recorded thereon for enabling a processor to quantify DNA mixture samples. In one example, the computer product comprises a computer readable medium having a computer executable or compilable logic (e.g., instructions) recorded thereon for enabling a processor to determine sources of fetal cellular DNA and/or use the fetal cellular DNA to determine fetal genetic conditions.

The sequence information from the sample under consideration may be mapped to chromosome reference sequences to identify a number of sequence tags for each of any one or more chromosomes of interest. In various embodiments, the reference sequences are stored in a database such as a relational or object database, for example.

It should be understood that it is not practical, or even possible in most cases, for an unaided human being to perform the computational operations of the methods disclosed herein. For example, mapping a single 30 bp read from a sample to any one of the human chromosomes might require years of effort without the assistance of a computational apparatus.

The methods disclosed herein can be performed using a system for quantifying DNA mixture samples. The system comprising: (a) a sequencer for receiving nucleic acids from the test sample providing nucleic acid sequence information from the sample; (b) a processor; and (c) one or more computer-readable storage media having stored thereon instructions for execution on said processor to carry out a method for determining sources of fetal cellular DNA and/or using the fetal cellular DNA to determine fetal genetic conditions.

In some embodiments, the methods are instructed by a computer-readable medium having stored thereon computer-readable instructions for carrying out a method for quantifying DNA mixture samples. Thus one embodiment provides a computer program product comprising one or more computer-readable non-transitory storage media having stored thereon computer-executable instructions that, when executed by one or more processors of a computer system, cause the computer system to implement a method for determining sources of fetal cellular DNA and/or using the fetal cellular DNA to determine fetal genetic conditions. The method includes: (a) receiving a genotype of the fetus in the current pregnancy, wherein the genotype of the fetus in the current pregnancy comprises one or more alleles for each genetic marker of a plurality of genetic markers, where each genetic marker represents a polymorphism at a unique genomic locus; (b) receiving a genotype of the pregnant female, wherein the genotype of the pregnant female comprises one or more alleles for each genetic marker of the plurality of the genetic markers; (c) identifying, from the genotype of the pregnant female and from the genotype of fetus in the current pregnancy, a set of informative genetic markers, wherein each informative genetic marker of the set of informative genetic markers is homozygous in the pregnant female and is heterozygous in the fetus in the current pregnancy; (d) for the fetal cellular DNA obtained from the pregnant female, determining one or more alleles at each informative genetic marker of the set of informative genetic markers, wherein the fetal cellular DNA originates from the fetus in the current pregnancy or a fetus in a historical pregnancy; (e) providing as input to a probabilistic model the one or more alleles at each informative genetic marker of the fetal cellular DNA obtained from the pregnant female; (f) obtaining as output of the probabilistic model probabilities of three scenarios: the fetal cellular DNA obtained from the pregnant female originates from a fetus in (1) the current pregnancy, (2) the historical pregnancy and having a same father as the fetus in the current pregnancy, and (3) the historical pregnancy and having a different father from the fetus in the current pregnancy; and (g) determining, from the output of the probabilistic model, whether the fetal cellular DNA originates from the fetus in (1) the current pregnancy. At least (e) and (f) are performed by a computer including a processor and memory.

In some embodiments, the instructions may further include automatically recording information pertinent to the method in a patient medical record for a human subject providing the test sample. The patient medical record may be maintained by, for example, a laboratory, physician's office, a hospital, a health maintenance organization, an insurance company, or a personal medical record website. Further, based on the results of the processor-implemented analysis, the method may further involve prescribing, initiating, and/or altering treatment of a human subject from whom the test sample was taken. This may involve performing one or more additional tests or analyses on additional samples taken from the subject.

Disclosed methods can also be performed using a computer processing system which is adapted or configured to perform a method for determining sources of fetal cellular DNA and/or using the fetal cellular DNA to determine fetal genetic conditions. One embodiment provides a computer processing system, which is adapted or configured to perform a method as described herein. In one embodiment, the apparatus comprises a sequencing device adapted or configured for sequencing at least a portion of the nucleic acid molecules in a sample to obtain the type of sequence information described elsewhere herein. The apparatus may also include components for processing the sample. Such components are described elsewhere herein.

Sequence or other data, can be input into a computer or stored on a computer readable medium either directly or indirectly. In one embodiment, a computer system is directly coupled to a sequencing device that reads and/or analyzes sequences of nucleic acids from samples. Sequences or other information from such tools are provided via interface in the computer system. Alternatively, the sequences processed by system are provided from a sequence storage source such as a database or other repository. Once available to the processing apparatus, a memory device or mass storage device buffers or stores, at least temporarily, sequences of the nucleic acids. In addition, the memory device may store tag counts for various chromosomes or genomes, etc. The memory may also store various routines and/or programs for analyzing the presenting the sequence or mapped data. Such programs/routines may include programs for performing statistical analyses, etc.

In one example, a user provides a sample into a sequencing apparatus. Data is collected and/or analyzed by the sequencing apparatus, which is connected to a computer. Software on the computer allows for data collection and/or analysis. Data can be stored, displayed (via a monitor or other similar device), and/or sent to another location. The computer may be connected to the internet which is used to transmit data to a handheld device utilized by a remote user (e.g., a physician, scientist or analyst). It is understood that the data can be stored and/or analyzed prior to transmittal. In some embodiments, raw data is collected and sent to a remote user or apparatus that will analyze and/or store the data. Transmittal can occur via the internet, but can also occur via satellite or other connection. Alternately, data can be stored on a computer-readable medium and the medium can be shipped to an end user (e.g., via mail). The remote user can be in the same or a different geographical location including, but not limited to a building, city, state, country or continent.

In some embodiments, the methods also include collecting data regarding a plurality of polynucleotide sequences (e.g., reads, tags and/or reference chromosome sequences) and sending the data to a computer or other computational system. For example, the computer can be connected to laboratory equipment, e.g., a sample collection apparatus, a nucleotide amplification apparatus, a nucleotide sequencing apparatus, or a hybridization apparatus. The computer can then collect applicable data gathered by the laboratory device. The data can be stored on a computer at any step, e.g., while collected in real time, prior to the sending, during or in conjunction with the sending, or following the sending. The data can be stored on a computer-readable medium that can be extracted from the computer. The data collected or stored can be transmitted from the computer to a remote location, e.g., via a local network or a wide area network such as the internet. At the remote location various operations can be performed on the transmitted data as described below.

Among the types of electronically formatted data that may be stored, transmitted, analyzed, and/or manipulated in systems, apparatus, and methods disclosed herein are the following:
Reads obtained by sequencing nucleic acids in a test sample
Tags obtained by aligning reads to a reference genome or other reference sequence or sequences
The reference genome or sequence
Allele counts—Counts or numbers of tags for each allele
Counts of shared genetic markers
Diagnoses (clinical condition associated with the calls)
Recommendations for further tests derived from the calls and/or diagnoses
Treatment and/or monitoring plans derived from the calls and/or diagnoses

These various types of data may be obtained, stored transmitted, analyzed, and/or manipulated at one or more locations using distinct apparatus. The processing options span a wide spectrum. At one end of the spectrum, all or much of this information is stored and used at the location where the test sample is processed, e.g., a doctor's office or other clinical setting. In other extreme, the sample is obtained at one location, it is processed and optionally sequenced at a different location, reads are aligned and calls are made at one or more different locations, and diagnoses, recommendations, and/or plans are prepared at still another location (which may be a location where the sample was obtained).

In various embodiments, the reads are generated with the sequencing apparatus and then transmitted to a remote site where they are processed to produce calls. At this remote location, as an example, the reads are aligned to a reference sequence to produce tags, which are counted and assigned to chromosomes or segments of interest. Also at the remote location, the doses are used to generate calls.

Among the processing operations that may be employed at distinct locations are the following:
Sample collection
Sample processing preliminary to sequencing
Sequencing
Analyzing sequence data and quantifying DNA mixture samples
Diagnosis
Reporting a diagnosis and/or a call to patient or health care provider
Developing a plan for further treatment, testing, and/or monitoring
Executing the plan
Counseling

Any one or more of these operations may be automated as described elsewhere herein. Typically, the sequencing and the analyzing of sequence data and quantifying DNA samples will be performed computationally. The other operations may be performed manually or automatically.

Examples of locations where sample collection may be performed include health practitioners' offices, clinics, patients' homes (where a sample collection tool or kit is provided), and mobile health care vehicles. Examples of locations where sample processing prior to sequencing may be performed include health practitioners' offices, clinics, patients' homes (where a sample processing apparatus or kit is provided), mobile health care vehicles, and facilities of DNA analysis providers. Examples of locations where sequencing may be performed include health practitioners' offices, clinics, health practitioners' offices, clinics, patients' homes (where a sample sequencing apparatus and/or kit is provided), mobile health care vehicles, and facilities of DNA analysis providers. The location where the sequencing takes place may be provided with a dedicated network connection for transmitting sequence data (typically reads) in an electronic format. Such connection may be wired or wireless and have and may be configured to send the data to a site where the data can be processed and/or aggregated prior to transmission to a processing site. Data aggregators can be maintained by health organizations such as Health Maintenance Organizations (HMOs).

The analyzing and/or deriving operations may be performed at any of the foregoing locations or alternatively at a further remote site dedicated to computation and/or the service of analyzing nucleic acid sequence data. Such locations include for example, clusters such as general purpose server farms, the facilities of a DNA analysis service business, and the like. In some embodiments, the computational apparatus employed to perform the analysis is leased or rented. The computational resources may be part of an internet accessible collection of processors such as processing resources colloquially known as the cloud. In some cases, the computations are performed by a parallel or massively parallel group of processors that are affiliated or unaffiliated with one another. The processing may be accomplished using distributed processing such as cluster computing, grid computing, and the like. In such embodiments, a cluster or grid of computational resources collective form a super virtual computer composed of multiple processors or computers acting together to perform the analysis and/or derivation described herein. These technologies as well as more conventional supercomputers may be employed to process sequence data as described herein. Each is a form of parallel computing that relies on processors or computers. In the case of grid computing these processors (often whole computers) are connected by a network (private, public, or the Internet) by a conventional network protocol such as Ethernet. By contrast, a supercomputer has many processors connected by a local high-speed computer bus.

In certain embodiments, the diagnosis is generated at the same location as the analyzing operation. In other embodiments, it is performed at a different location. In some examples, reporting the diagnosis is performed at the location where the sample was taken, although this need not be the case. Examples of locations where the diagnosis can be generated or reported and/or where developing a plan is performed include health practitioners' offices, clinics, internet sites accessible by computers, and handheld devices such as cell phones, tablets, smart phones, etc. having a wired or wireless connection to a network. Examples of locations where counseling is performed include health practitioners' offices, clinics, internet sites accessible by computers, handheld devices, etc.

In some embodiments, the sample collection, sample processing, and sequencing operations are performed at a first location and the analyzing and deriving operation is performed at a second location. However, in some cases, the sample collection is collected at one location (e.g., a health practitioner's office or clinic) and the sample processing and sequencing is performed at a different location that is optionally the same location where the analyzing and deriving take place.

In various embodiments, a sequence of the above-listed operations may be triggered by a user or entity initiating sample collection, sample processing and/or sequencing. After one or more these operations have begun execution the other operations may naturally follow. For example, the sequencing operation may cause reads to be automatically collected and sent to a processing apparatus which then conducts, often automatically and possibly without further user intervention, the sequence analysis and quantifying DNA mixture samples. In some implementations, the result of this processing operation is then automatically delivered, possibly with reformatting as a diagnosis, to a system component or entity that processes reports the information to a health professional and/or patient. As explained such information can also be automatically processed to produce a treatment, testing, and/or monitoring plan, possibly along with counseling information. Thus, initiating an early stage operation can trigger an end to end sequence in which the health professional, patient or other concerned party is provided with a diagnosis, a plan, counseling and/or other information useful for acting on a physical condition. This is accomplished even though parts of the overall system are physically separated and possibly remote from the location of, e.g., the sample and sequence apparatus.

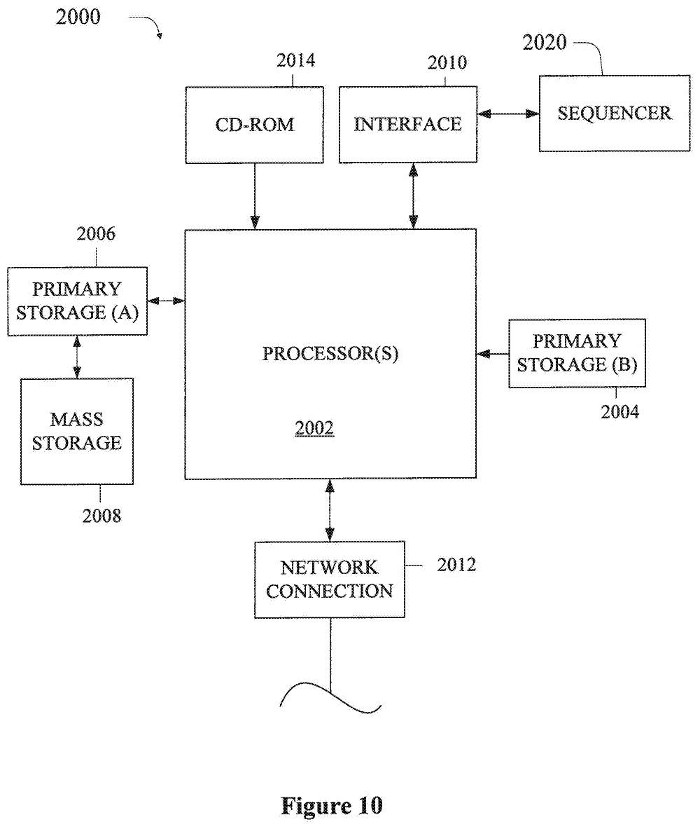

FIG. 10 illustrates, in simple block format, a typical computer system that, when appropriately configured or designed, can serve as a computational apparatus according to certain embodiments. The computer system 2000 includes any number of processors 2002 (also referred to as central processing units, or CPUs) that are coupled to storage devices including primary storage 2006 (typically a random access memory, or RAM), primary storage 2004 (typically a read only memory, or ROM). CPU 2002 may be of various types including microcontrollers and microprocessors such as programmable devices (e.g., CPLDs and FPGAs) and non-programmable devices such as gate array ASICs or general-purpose microprocessors. In the depicted embodiment, primary storage 2004 acts to transfer data and instructions uni-directionally to the CPU and primary storage 2006 is used typically to transfer data and instructions in a bi-directional manner. Both of these primary storage devices may include any suitable computer-readable media such as those described above. A mass storage device 2008 is also coupled bi-directionally to primary storage 2006 and provides additional data storage capacity and may include any of the computer-readable media described above. Mass storage device 2008 may be used to store programs, data and the like and is typically a secondary storage medium such as a hard disk. Frequently, such programs, data and the like are temporarily copied to primary memory 2006 for execution on CPU 2002. It will be appreciated that the information retained within the mass storage device 2008, may, in appropriate cases, be incorporated in standard fashion as part of primary storage 2004. A specific mass storage device such as a CD-ROM 2014 may also pass data uni-directionally to the CPU or primary storage.

CPU 2002 is also coupled to an interface 2010 that connects to one or more input/output devices such as such as a nucleic acid sequencer (2020), video monitors, track balls, mice, keyboards, microphones, touch-sensitive displays, transducer card readers, magnetic or paper tape readers, tablets, styluses, voice or handwriting recognition peripherals, USB ports, or other well-known input devices such as, of course, other computers. Finally, CPU 2002 optionally may be coupled to an external device such as a database or a computer or telecommunications network using an external connection as shown generally at 2012. With such a connection, it is contemplated that the CPU might receive information from the network, or might output information to the network in the course of performing the method steps described herein. In some implementations, a nucleic acid sequencer (2020) may be communicatively linked to the CPU 2002 via the network connection 2012 instead of or in addition to via the interface 2010.

In one embodiment, a system such as computer system 2000 is used as a data import, data correlation, and querying system capable of performing some or all of the tasks described herein. Information and programs, including data files can be provided via a network connection 2012 for access or downloading by a researcher. Alternatively, such information, programs and files can be provided to the researcher on a storage device.

In a specific embodiment, the computer system 2000 is directly coupled to a data acquisition system such as a microarray, high-throughput screening system, or a nucleic acid sequencer (2020) that captures data from samples. Data from such systems are provided via interface 2010 for analysis by system 2000. Alternatively, the data processed by system 2000 are provided from a data storage source such as a database or other repository of relevant data. Once in apparatus 2000, a memory device such as primary storage 2006 or mass storage 2008 buffers or stores, at least temporarily, relevant data. The memory may also store various routines and/or programs for importing, analyzing and presenting the data, including sequence reads, UMIs, codes for determining sequence reads, collapsing sequence reads and correcting errors in reads, etc.

In certain embodiments, the computers used herein may include a user terminal, which may be any type of computer (e.g., desktop, laptop, tablet, etc.), media computing platforms (e.g., cable, satellite set top boxes, digital video recorders, etc.), handheld computing devices (e.g., PDAs, e-mail clients, etc.), cell phones or any other type of computing or communication platforms.

In certain embodiments, the computers used herein may also include a server system in communication with a user terminal, which server system may include a server device or decentralized server devices, and may include mainframe computers, mini computers, super computers, personal computers, or combinations thereof. A plurality of server systems may also be used without departing from the scope of the present invention. User terminals and a server system may communicate with each other through a network. The network may comprise, e.g., wired networks such as LANs (local area networks), WANs (wide area networks), MANs (metropolitan area networks), ISDNs (Intergrated Service Digital Networks), etc. as well as wireless networks such as wireless LANs, CDMA, Bluetooth, and satellite communication networks, etc. without limiting the scope of the present invention.

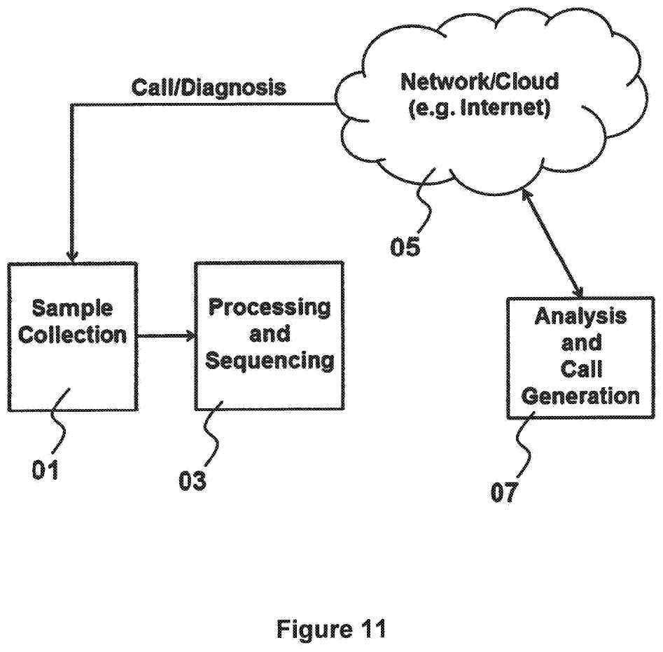

FIG. 11 shows one implementation of a dispersed system for producing a call or diagnosis from a test sample. A sample collection location 01 is used for obtaining a test sample from a patient such as a pregnant female or a putative cancer patient. The samples then provided to a processing and sequencing location 03 where the test sample may be processed and sequenced as described above. Location 03 includes apparatus for processing the sample as well as apparatus for sequencing the processed sample. The result of the sequencing, as described elsewhere herein, is a collection of reads which are typically provided in an electronic format and provided to a network such as the Internet, which is indicated by reference number 05 in FIG. 11.

The sequence data is provided to a remote location 07 where analysis and call generation are performed. This location may include one or more powerful computational devices such as computers or processors. After the computational resources at location 07 have completed their analysis and generated a call from the sequence information received, the call is relayed back to the network 05. In some implementations, not only is a call generated at location 07 but an associated diagnosis is also generated. The call and or diagnosis are then transmitted across the network and back to the sample collection location 01 as illustrated in FIG. 11. As explained, this is simply one of many variations on how the various operations associated with generating a call or diagnosis may be divided among various locations. One common variant involves providing sample collection and processing and sequencing in a single location. Another variation involves providing processing and sequencing at the same location as analysis and call generation.

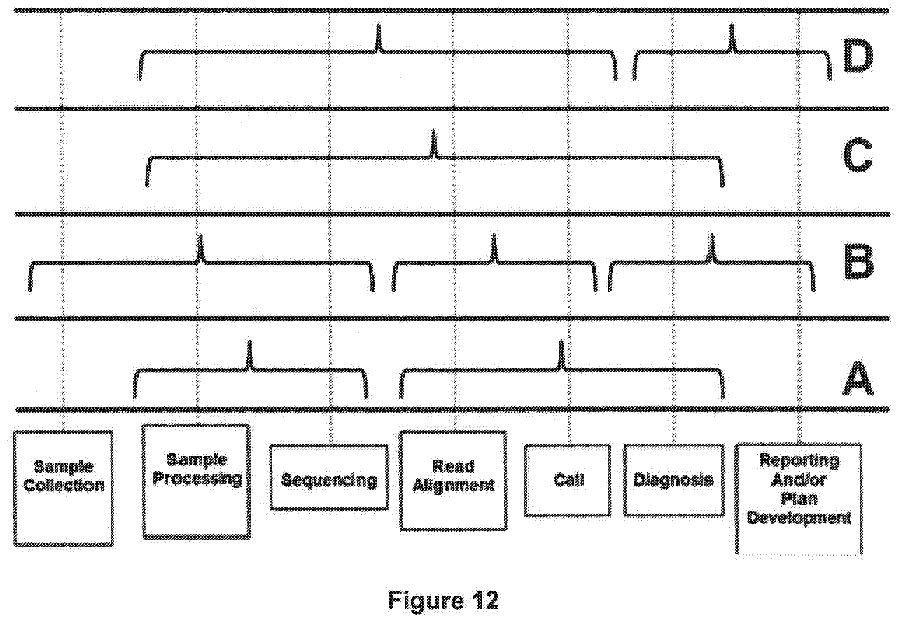

FIG. 12 elaborates on the options for performing various operations at distinct locations. In the most granular sense depicted in FIG. 12, each of the following operations is performed at a separate location: sample collection, sample processing, sequencing, read alignment, calling, diagnosis, and reporting and/or plan development.

In one embodiment that aggregates some of these operations, sample processing and sequencing are performed in one location and read alignment, calling, and diagnosis are performed at a separate location. See the portion of FIG. 12 identified by reference character A. In another implementation, which is identified by character B in FIG. 12, sample collection, sample processing, and sequencing are all performed at the same location. In this implementation, read alignment and calling are performed in a second location. Finally, diagnosis and reporting and/or plan development are performed in a third location. In the implementation depicted by character C in FIG. 12, sample collection is performed at a first location, sample processing, sequencing, read alignment, calling, and diagnosis are all performed together at a second location, and reporting and/or plan development are performed at a third location. Finally, in the implementation labeled D in FIG. 12, sample collection is performed at a first location, sample processing, sequencing, read alignment, and calling are all performed at a second location, and diagnosis and reporting and/or plan management are performed at a third location.

One embodiment provides a system for analyzing cell-free DNA (cfDNA) for simple nucleotide variants associated with tumors, the system including a sequencer for receiving a nucleic acid sample and providing nucleic acid sequence information from the nucleic acid sample; a processor; and a machine readable storage medium comprising instructions for execution on said processor, the instructions comprising: code for mapping the nucleic acid sequence reads to one or more polymorphism loci on a reference sequence; code for determining, using the mapped nucleic acid sequence reads, allele counts of nucleic acid sequence reads for one or more alleles at the one or more polymorphism loci; and code for quantifying, using a probabilistic mixture model, one or more fractions of nucleic acid of the one or more contributors in the nucleic acid sample, wherein using the probabilistic mixture model comprises applying a probabilistic mixture model to the allele counts of nucleic acid sequence reads, and the probabilistic mixture model uses probability distributions to model the allele counts of nucleic acid sequence reads at the one or more polymorphism loci, the probability distributions accounting for errors in the nucleic acid sequence reads.

In some embodiments of any of the systems provided herein, the sequencer is configured to perform next generation sequencing (NGS). In some embodiments, the sequencer is configured to perform massively parallel sequencing using sequencing-by-synthesis with reversible dye terminators. In other embodiments, the sequencer is configured to perform sequencing-by-ligation. In yet other embodiments, the sequencer is configured to perform single molecule sequencing.

### Example

### Setup

This example uses implementations of the disclosed methods to determine sources of fetal cellular DNA using simulation data. The example collects a set of n informative loci, i.e. where mother is homozygous and the cfDNA indicates the fetus has at least one non-maternal allele.

The method simulates the non-maternal allele frequency (hetero-allele frequency) with a uniform distribution. When applied to real data, for each of the j loci, the non-maternal allele frequency pᵢ is the population frequency of that allele. When applied to actual test data, the set of informative loci used in any experiment is dynamic. Their allele frequency can be provided to the process.

n.informative.loci <-512
non.maternal.allele.frequency <-runif(n.informative.loci)

### Model Description

Let s denote a paternal relationship scenario then for each of the i scenarios under consideration calculate

$$p ( {s}_{i} \vert k ) = \frac{p ( k \vert {s}_{i} ) p ( {s}_{i} )}{p ( k )} \qquad ( Eq . 1 )$$

The most likely parental relationship scenario from the set considered is the one with the highest posterior probability.

### Likelihood Function

The likelihood function is given by the beta binomial distribution

$$p ( k \vert {s}_{i} ) = ( \begin{matrix}n \\ k\end{matrix} ) \frac{B ( k + {a}_{i} , n - k + {b}_{i} )}{B ( {a}_{i} , {b}_{i} )} \qquad ( Eq . 5 )$$

The beta binomial distribution is a compound distribution which models the number of matching alleles k as a random variable drawn from a binomial distribution with a success rate μ, which is itself a random variable drawn from a beta distribution with hyperparameters a and b.

This function is implemented in the following way, which returns probabilities on log scale to prevent underflow.

beta.binom.pmf <− function(k,n,a,b){

return(1choose(n,k) + 1beta(k+a, n−k+b) − 1beta(a, b))

}

For each scenario, the hyperparameters a and b is set in the following way.

aᵢ=μᵢ*w (Eq. 6)

bᵢ=(1−μᵢ)*w (Eq. 7)

where μᵢ corresponds to proportion of loci which are expected to match under the i^{th} scenario.

The w parameter is interpreted as a number of pseudo counts and determines the concentration of the prior distribution around values corresponding to μ.

Modelling the expected number of matches in this way allows for the model to be robust to measurement errors as well as errors in the calculation of μ for each scenario. Errors in the calculation of μ could arise due to errors in the publically available tables of allele frequencies for members of the set of informative loci.

Scenario (1): Same Fetus

When the fetal cell comes from the same fetus as the cfDNA, all informative markers should have a non-maternal hetero-allele. However for computational reasons, the following expression is used.

$${μ}_{1} = 1 - \frac{1}{n + 1} \qquad ( Eq . 8 )$$

Scenario (2): Different Fetus, Same Father

Under the assumption that the samples come from different fetuses that share the same father, then by definition the father must have at least 1 copy of the hetero-allele at each informative locus.

If at the j^{th} locus, the father's second allele is also a hetero-allele, then a match will always occur. The probability that the second allele is also a hetero-allele is pⱼ, Assuming the father is not a product of inbreeding.

When the father's remaining allele is not a hetero-allele, which occurs with probability 1−pⱼ, then a match will only occur if the hetero-allele is passed on by chance due to random segregation, adding a factor of ½. Summing over all informative loci, this leads to the following expression for μ₂.

$${μ}_{2} = \frac{1}{n} {\sum }_{j = 1}^{n} [ {p}_{j} + \frac{1}{2} ( 1 - {p}_{j} ) ] \qquad ( Eq . 9 )$$

Scenario (3): Different Fetus Different Fathers

Under the assumption that there is no relationship between the fathers of the two fetuses, the fetal cell should only have hetero-alleles at informative loci at a frequency determined by the population allele frequency.

The father of the cFC sample can have either 0, 1 or 2 copies of the hetero-allele. A match occurs when there are 2 copies, which should occur with probability pⱼ², or when there is one copy, which should occur with probability 2pⱼ(1−pⱼ), and when that copy is passed on by chance due to random segregation, adding a factor of ½. Summing over all informative loci, this leads to the following expression for the expected number of matches.

$${μ}_{3} = \frac{1}{n} {\sum }_{j = 1}^{n} {p}_{j}^{2} + \frac{1}{2} ( 2 {p}_{j} ( 1 - {p}_{j} ) )$$

which simplifies to the mean population frequency of the set of loci

$${μ}_{3} = \frac{1}{n} {\sum }_{j = 1}^{n} {p}_{j} \qquad ( Eq . 10 )$$

Priors Over Scenarios p(sᵢ)

In this example we assume a uniform prior over each of the scenarios. In implementations applied to actual test subjects, the priors could be functions of any relevant information about the relative frequency. For example, the prior may be implemented as a function of number of previous pregnancies, time since last pregnancy, etc.

Calculation of p(k)

The normalizing constant p(k) is given by

p(k)=Σᵢp(k|sᵢ)p(sᵢ) (11)

The outputs of the likelihood function for each scenario were log scaled to avoid underflow. To normalize likelihoods and calculate posteriors this function is used to normalize in log scale and then returns probabilities on the conventional scale.

logp2p <− function(x){

xd = x − max(x)
exd = exp(xd)
return(exd/sum(exd))

}

### Calculation Steps Pseudocode

d <− data.frame(scenarios=c(“Same Fetus”,

“Different Fetus Same Father”,
“Different Fathers”),

n.matches.expected = c(n.informative.loci,

sum(non.maternal.allele.freque

ncy +0.5*(1-non.maternal.allele.frequency)) ,

sum(non.maternal.allele.freque

ncy))

)

d$mu <− c(1 − 1/(1+n.informative.loci),

d$n.matches.expected[2]/n.informative.loci,
d$n. matches.expected[3]/n.informative.loci)

d

| ## | scenarios n.matches.expected mu | |
|---|---|---|
| ## 1 | Same Fetus | 512.0000 0.9980507 |

| ## 2 Different Fetus Same Father | | 382.2887 0.7466576 |
|---|---|---|
| ## 3 | Different Fathers | 252.5774 0.4933152 |

Set the hyperperameter w to correspond 16 pseudo observations.

w<-16

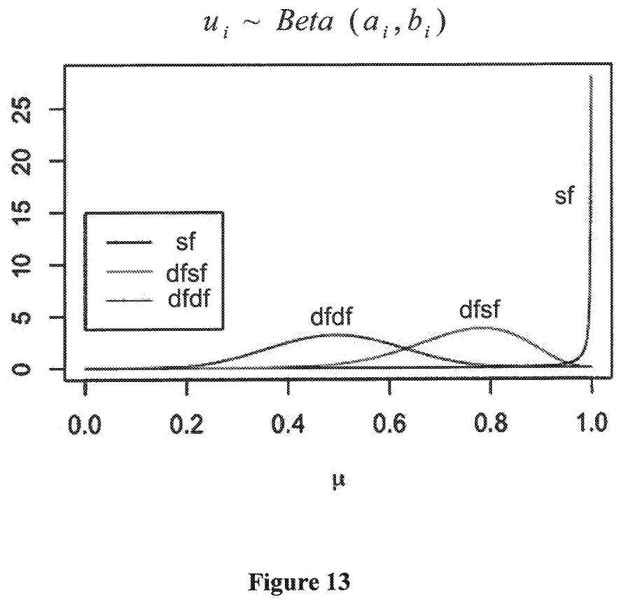

FIG. 13 illustrates uᵢ˜Beta(aᵢ,bᵢ), which are the beta distributions of the expected portion of shared genetic markers (p) for the three different scenarios: (1) same fetus, (2) different fetuses and same father, and (3) different fetuses and different fathers. The distribution for scenario (1) has a mode near 1. The distribution for scenario (2) has a mode near 0.75. The distribution for scenario (3) has a mode near 0.5.

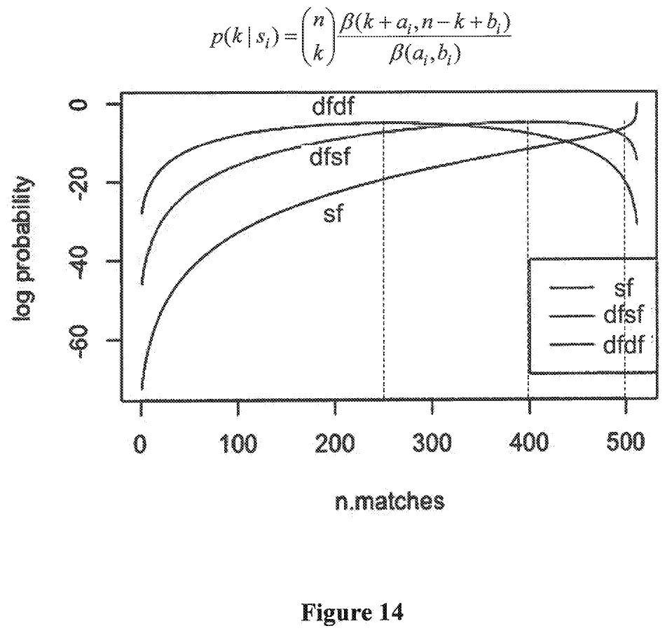

FIG. 14 illustrates log probability as a function of number of shared/matched genetic markers. Each curve represents one of the three scenarios. The log probability is shown on the y-axis. The number of shared genetic markers is shown on the x-axis. For example, when 250 shared genetic markers are observed in the test data, the log probability for the scenario (3)—different fetuses and different fathers—is the highest, as illustrated by the vertical line one the left. When 400 shared genetic markers are observed in the test data, the log probability for the scenario (2)—different fetuses and same father—is the highest, as illustrated by the vertical line in the middle. When 500 shared genetic markers are observed in the test data, the log probability for the scenario (1)—same fetus—is the highest, as illustrated by the vertical line on the right.

### Example Posterior Calculation Pseudocode

Assume we have established n=512 informative loci between maternal genotypes and cfDNA non-maternal hetero-allales. We then observe a fetal cell which has non-maternal hetero-alleles at 500 of the informative loci, what is the probability this cell came from the same fetus as the cfDNA?

n.matches.observed <− 500
d$posterior <− c(0,0,0)
for (i in 1:3)
{
d$posterior[i] = beta.binom.pmf(n.matches.observed, n.informative.1
oci, d$mu[i]*w, (1-d$mu[i])*w)
}
d$posterior <− round(logp2p(d$posterior), 2)
d

| | | |
|---|---|---|
| ## | scenarios n.matches.expected | mu posteri |

or

| | | | |
|---|---|---|---|
| ## 1 | Same Fetus | 512.0000 0.9980507 | 0. |

93

| | | |
|---|---|---|
| ## 2 Different Fetus Same Father | 382.2887 0.7466576 | 0. |

07

| | | | |
|---|---|---|---|
| ## 3 | Different Fathers | 252.5774 0.4933152 | 0. |

00

When 500 shared genetic markers are observed in the test data, posterior probability for scenario (1) is 0.98, scenario (2) is 0.07, and scenario (3) is 0. As such, the method determines that the cFC is from the same fetus providing the cfDNA.

Although the foregoing invention has been described in some detail for purposes of clarity of understanding, it will be apparent that certain changes and modifications may be practiced within the scope of the invention. It should be noted that there are many alternative ways of implementing the processes and databases of the present invention. Accordingly, the present embodiments are to be considered as illustrative and not restrictive, and the invention is not to be limited to the details given herein.

What is claimed is:

1. A method of determining a genetic origin of fetal cellular DNA obtained from a pregnant female who is carrying a fetus in a current pregnancy, the method comprising:
(a) receiving a genotype of the fetus in the current pregnancy, wherein the genotype of the fetus in the current pregnancy comprises one or more alleles for each genetic marker of a plurality of genetic markers, where each genetic marker represents a polymorphism at a unique genomic locus;
(b) receiving a genotype of the pregnant female, wherein the genotype of the pregnant female comprises one or more alleles for each genetic marker of the plurality of the genetic markers;
(c) identifying, from the genotype of the pregnant female and from the genotype of fetus in the current pregnancy, a set of informative genetic markers, wherein each informative genetic marker of the set of informative genetic markers is homozygous in the pregnant female and is heterozygous in the fetus in the current pregnancy;
(d) for the fetal cellular DNA obtained from the pregnant female, determining one or more alleles at each informative genetic marker of the set of informative genetic markers, wherein the fetal cellular DNA originates from the fetus in the current pregnancy or a fetus in a historical pregnancy;
(e) providing as input to a probabilistic model the one or more alleles at each informative genetic marker of the fetal cellular DNA obtained from the pregnant female;
(f) obtaining, as output of the probabilistic model, a probability that the fetal cellular DNA obtained from the pregnant female originates from a fetus in the current pregnancy; and
(g) determining, from the output of the probabilistic model, whether the fetal cellular DNA originates from the fetus in the current pregnancy,
wherein at least (e) and (f) are performed by a computer comprising a processor and memory.

2. The method of claim 1, wherein (f) comprises: obtaining, as output of the probabilistic model, probabilities of three scenarios: the fetal cellular DNA obtained from the pregnant female originates from a fetus in (1) the current pregnancy, (2) the historical pregnancy and having a same father as the fetus in the current pregnancy, and (3) the historical pregnancy and having a different father from the fetus in the current pregnancy.

3. The method of claim 2, wherein (g) comprises: determining whether the fetal cellular DNA originates from the fetus in (1) the current pregnancy, (2) the historical pregnancy and having a same father as the fetus in current pregnancy, or (3) the historical pregnancy and having a different father as the fetus in the current pregnancy.

4. The method of claim 2, wherein (e) comprises providing as input to the probabilistic model a number of shared genetic markers, wherein a shared genetic marker is a genetic marker in the informative genetic markers for which the fetal cellular DNA obtained from the pregnant female and the fetus in the current pregnancy have same alleles.

5. The method of claim 4, wherein the probabilistic model calculates the probabilities of the three scenarios given the number of shared genetic markers based on probabilities of the number of shared genetic markers given the three scenarios.

6. The method of claim 5, wherein the probabilistic model calculates the probabilities of the three scenarios given the number of shared genetic markers as follows:
$$p ( {s}_{i} \vert k ) = \frac{p ( k \vert {s}_{i} ) p ( {s}_{i} )}{p ( k )}$$
wherein
p(sᵢ|k) is a probability of scenario i, or sᵢ, given the number of shared genetic markers, or k,
p(k|sᵢ) is a probability of the number of shared genetic markers given scenario i,
p(sᵢ) is an overall probability of scenario i, and
p(k) is an overall probability of the number of shared genetic markers.

7. The method of any of claims 5-6, wherein, for each scenario, the probabilistic model simulates the number of shared genetic markers given scenario i, or k|sᵢ, as a random variable drawn from a beta-binomial distribution.

8. The method of claim 7, wherein the probabilistic model simulates the number of shared genetic markers given scenario i, or k|sᵢ as a random variable drawn from a binomial distribution with a success rate μᵢ, and μᵢ is a random variable drawn from a beta distribution with hyperparameters aᵢ and bᵢ; namely, k|sᵢ˜BN(n,μᵢ) and μᵢ˜Beta(aᵢ,bᵢ), n being the number of informative genetic markers in the set of informative genetic markers.

9. The method of claim 8, wherein the probability of the number of shared genetic markers given scenario i is calculated from the following likelihood function:
$$p ( k \vert {s}_{i} ) = ( \begin{matrix}n \\ k\end{matrix} ) \frac{B ( k + {a}_{i} , n - k + {b}_{i} )}{B ( {a}_{i} , {b}_{i} )}$$
wherein
n is the number of informative genetic markers,
k is the number of shared genetic markers,
β( ) is a beta function, and
aᵢ and bᵢ are the hyperparameters of the beta distribution for scenario i.

10. The method of any of claims 8-9, wherein
aᵢ=μᵢ*w
bᵢ=(1−μᵢ)*w
wherein w is a parameter representing a number of pseudo counts or observations.

11. The method of any of claims 8-10, wherein μᵢ is set to correspond to an expected proportion of shared genetic markers among the set of informative genetic markers in scenario i.

12. The method of claim 11, wherein the probabilistic model calculates μ₁, the expected proportion of shared genetic markers for scenario (1), as follows:
$${μ}_{1} = 1 - \frac{1}{n + 1}$$
wherein n is the number of informative genetic markers.

13. The method of claim 11, wherein the probabilistic model calculates μ₂, the expected proportion of shared genetic markers for scenario (2), as follows,
$${μ}_{2} = \frac{1}{n} {\sum }_{j = 1}^{n} [ {p}_{j} + \frac{1}{2} ( 1 - {p}_{j} ) ]$$
wherein pᵢ is a population frequency of a hetero-allele at the j^{th} marker, the hetero-allele being an allele at an informative genetic marker found in the fetus in the current pregnancy but not in the pregnant female.

14. The method of claim 11, wherein the probabilistic model calculates μ₃, the expected proportion of shared genetic markers for scenario (3), as follows:
$${μ}_{3} = \frac{1}{n} {\sum }_{j = 1}^{n} {p}_{j}$$
wherein
pⱼ is a population frequency of a hetero-allele at the j^{th} marker.

15. The method of claim 2, further comprising providing prior probabilities of the three scenarios to the probabilistic model, wherein the probabilistic model provides posterior probabilities of the three scenarios based on the prior probabilities of the three scenarios, as well as on the alleles at the one or more markers.

16. The method of any of the preceding claims, further comprising:
obtaining cell free DNA (“cfDNA”) from the pregnant female; and
genotyping the cfDNA from the pregnant female to produce (i) the genotype of the fetus in the current pregnancy, and (ii) the genotype of the pregnant female.

17. The method of any of the preceding claims, further comprising:
obtaining at least one cell of the pregnant female;
genotyping cellular DNA obtained from the at least one cell of the pregnant female to produce the genotype of the pregnant female;
obtaining cfDNA from the pregnant female; and
genotyping the cfDNA from the pregnant female to produce the genotype of the fetus in the current pregnancy.

18. The method of any of the preceding claims, wherein the fetal cellular DNA is from a circulating fetal cell (“cFC”) circulating in the pregnant female.

19. The method of claim 18, further comprising determining a genetic origin of the cFC.

20. The method of any of the preceding claims, wherein the fetal cellular DNA is determined to originate from the fetus in the current pregnancy, and the method further comprises analyzing the fetal cellular DNA to determine whether the fetus in the current pregnancy has a genetic abnormality.

21. The method of claim 20, wherein the genetic abnormality is an aneuploidy.

22. The method of claim 20, wherein the analyzing the fetal cellular DNA comprises using both information from the fetal cellular DNA and information from fetal cfDNA obtained from the pregnant female during the current pregnancy to determine whether the fetus in the current pregnancy has the genetic abnormality.

23. The method of any of the preceding claims, wherein each informative genetic marker is biallelic.

24. A computer program product comprising a non-transitory machine readable medium storing program code that, when executed by one or more processors of a computer system, causes the computer system to implement a method of determining the genetic origin of fetal cellular DNA obtained from a pregnant female who is carrying a fetus in a current pregnancy, said program code comprising:
(a) code for determining, for the fetal cellular DNA obtained from the pregnant female, one or more alleles at each informative genetic marker of a set of informative genetic markers,
wherein
each informative genetic marker represents a polymorphism at a unique genomic locus,
each informative genetic marker is homozygous in the pregnant female and is heterozygous in the fetus in the current pregnancy, and
the fetal cellular DNA originates from the fetus in the current pregnancy or a fetus in a historical pregnancy; and
(b) code for providing as input to a probabilistic model the one or more alleles at each informative genetic marker of the fetal cellular DNA obtained from the pregnant female;
(c) code for obtaining as output of the probabilistic model probabilities of three scenarios: the fetal cellular DNA obtained from the pregnant female originating from a fetus in (1) the current pregnancy, (2) the historical pregnancy and having a same father as the fetus in the current pregnancy, and (3) the historical pregnancy and having a different father from the fetus in the current pregnancy; and
(d) code for determining, from the output of the probabilistic model, whether the fetal cellular DNA originates from the fetus in (1) the current pregnancy.

25. A computer system, comprising:
one or more processors;
system memory; and
one or more computer-readable storage media having stored thereon computer-executable instructions that, when executed by the one or more processors, cause the computer system to implement a method of determining the genetic origin of fetal cellular DNA obtained from a pregnant female who is carrying a fetus in a current pregnancy, the method comprising:
(a) determining, for the fetal cellular DNA obtained from the pregnant female, one or more alleles at each informative genetic marker of to set of informative genetic markers,
wherein
each informative genetic marker represents a polymorphism at a unique genomic locus,
each informative genetic marker is homozygous in the pregnant female and is heterozygous in the fetus in the current pregnancy, and
the fetal cellular DNA originates from the fetus in the current pregnancy or a fetus in a historical pregnancy; and
(b) providing as input to a probabilistic model the one or more alleles at each informative genetic marker of the fetal cellular DNA obtained from the pregnant female;
(c) obtaining as output of the probabilistic model probabilities of three scenarios: the fetal cellular DNA obtained from the pregnant female originating from a fetus in (1) the current pregnancy, (2) the historical pregnancy and having a same father as the fetus in the current pregnancy, and (3) the historical pregnancy and having a different father from the fetus in the current pregnancy; and
(d) determining, from the output of the probabilistic model, whether the fetal cellular DNA originates from the fetus in (1) the current pregnancy.

26. A method for matching pairs of character strings using probabilistic modeling and computer simulation, wherein two character strings in any pair have a same number of characters, the method comprising:
(a) receiving a first pair of character strings;
(b) receiving a fifth pair of character strings;
(c) identifying a set of informative character positions in both the first pair of character strings and the fifth pair of character strings, wherein each informative character position of the set of informative character positions (i) represents a unique position in each character string, (ii) has one or both of two different characters in any pair of character strings, (iii) has only one character of said two different characters in the fifth pair of character strings, and (iv) has both characters of said two different characters in the first pair of character strings;
(d) determining, for a fourth pair of character strings, characters at the set of informative character positions;
(e) providing, as input to a probabilistic model, the characters at the set of informative character positions of the fourth pair of character strings, wherein the probabilistic model was trained using a training dataset comprising pairs of character strings;
(f) obtaining, as output of the probabilistic model, a probability that the fourth pair of character strings matches the first pair of character strings, wherein two different character strings of each pair of character strings have a same length, each informative character position has a corresponding position on each character strings, the first pair of character strings is obtainable by recombining the fifth pair of character strings with a sixth pair of pair of character strings; and
(g) determining, from the output of the probabilistic model, whether the fourth pair of character strings matches the first pair of character strings,
wherein at least (e) and (f) are performed by a computer system comprising a processor and memory.

27. The method of claim 26, wherein (f) comprises: obtaining probabilities of three scenarios: the fourth pair of character strings matches the first, a second, and a third pair of character strings, wherein the second pair of character strings is obtainable by recombining the fifth pair of character strings with the sixth pair of character strings, and the third pair of character strings is obtainable by recombining the fifth pair of character strings with a seventh pair of character strings.

28. The method of claim 27, wherein (g) comprises determining, from the output of the probabilistic model, whether the fourth pair of character strings matches the first, second, or third pair of character strings.
